SEQUENCE LISTING (31 sequences)

SEQ ID NO: 1 - protein, 9 aa, synthetic construct
  source 1..9: note = Wild type parent peptide 1.0, hotspot R157
EYGIQRITC

SEQ ID NO: 2 - protein, 10 aa, synthetic construct
  source 1..10: note = Wild type parent peptide 2.0, hotspot K238
VGSPKNAVPP

SEQ ID NO: 3 - protein, 10 aa, synthetic construct
  source 1..10: note = Wild type parent peptide 3.0, hotspot K343/K346
GEVAKAAKVK

SEQ ID NO: 4 - protein, 9 aa, synthetic construct
  source 1..9: note = Alanine Substitution Peptide 1.1, hotspot R157
EYGIQAITC

SEQ ID NO: 5 - protein, 10 aa, synthetic construct
  source 1..10: note = Alanine Substitution Peptide 2.1, hotspot K238
VGSPANAVPP

SEQ ID NO: 6 - protein, 10 aa, synthetic construct
  source 1..10: note = Alanine Substitution Peptide 3.1, hotspot K343/K346
GEVAAAAAVK

SEQ ID NO: 7 - protein, 9 aa, synthetic construct
  source 1..9: note = Scrambled Peptide 1.2, hotspot R157
RGQETCYII

SEQ ID NO: 8 - protein, 10 aa, synthetic construct
  source 1..10: note = Scrambled Peptide 2.2, hotspot K238
VNPSPVPAGK

SEQ ID NO: 9 - protein, 10 aa, synthetic construct
  source 1..10: note = Scrambled peptide 3.2, hotspot K343/K346
KAAEKVAGVK

SEQ ID NO: 10 - protein, 9 aa, synthetic construct
  source 1..9: note = Thioether cyclized peptide 1.3, first and last residues linked, Hotspot R157
EYGIQRITC

SEQ ID NO: 11 - protein, 11 aa, synthetic construct
  source 1..11: note = Thioether cyclized peptide 2.3, first and last residues linked, Hotspot K238
VGSPKNAVPPC

SEQ ID NO: 12 - protein, 11 aa, synthetic construct
  source 1..11: note = Thioether cyclized peptide 3.3, first and last residues linked, Hotspot K343/K346
GEVAKAAKVKC

SEQ ID NO: 13 - protein, 6 aa, synthetic construct
  source 1..6: note = Truncation peptide 1.4, Hotspot R157
EYGIRC

SEQ ID NO: 14 - protein, 5 aa, synthetic construct
  source 1..5: note = Truncation peptide 2.4, Hotspot K238
SPKNV

SEQ ID NO: 15 - protein, 7 aa, synthetic construct
  source 1..7: note = Truncation peptide 3.4, Hotspot K343/K346
VAKAAKV

SEQ ID NO: 16 - protein, 11 aa, synthetic construct
  source 1..11: note = Azidolysine peptide, Hotspot R157, disulfide link between terminal Cys residues
  MOD_RES 1: note = N terminal acetyl group
  MOD_RES 2: note = Azidolysine N3
  MOD_RES 11: note = C terminal amidation NH2
CKEYGIQRITC

SEQ ID NO: 17 - protein, 13 aa, synthetic construct
  source 1..13: note = Azidolysine peptide, Hotspot K238, thioether cycle between terminal Ala and Cys residues
  MOD_RES 1: note = N terminal acetyl group
  MOD_RES 2: note = Azidolysine N3
  MOD_RES 13: note = C terminal amidation NH2
AKVGSPKNAVPPC

SEQ ID NO: 18 - protein, 13 aa, synthetic construct
  source 1..13: note = Azidolysine peptide, Hotspot K343/K346, disulfide bond between terminal Cys residues
  MOD_RES 1: note = N terminal acetyl group
  MOD_RES 2: note = Azidolysine N3
  MOD_RES 13: note = C terminal amidation
CKGEVAKAAKVKC

SEQ ID NO: 19 - DNA, 78 bp, synthetic construct
  source 1..78: mol_type = other DNA; note = S1 Scaffold
tgggaaggcactttttcatggcttagcatgtggccaatttttccagtggctcaactagctgtctttttgtcagtctcc

SEQ ID NO: 20 - DNA, 21 bp, synthetic construct
  source 1..21: mol_type = other DNA; note = S2 Staple1
acccttccgtggtaccgaatc

SEQ ID NO: 21 - DNA, 21 bp, synthetic construct
  source 1..21: mol_type = other DNA; note = S3 Staple2
gtacaccggttggtcaccgag

SEQ ID NO: 22 - DNA, 21 bp, synthetic construct
  source 1..21: mol_type = other DNA; note = S4 Staple3
ttgatcgacagcagtcagagg

SEQ ID NO: 23 - DNA, 21 bp, synthetic construct
  source 1..21: mol_type = other DNA; note = S2 DBCO
  modified_base 21: mod_base = OTHER; note = Dibenzylcyclooctyne
acccttccgtggtaccgaatc

SEQ ID NO: 24 - DNA, 21 bp, synthetic construct
  source 1..21: mol_type = other DNA; note = S3 DBCO
  modified_base 21: mod_base = OTHER; note = Dibenzylcyclooctyne
gtacaccggttggtcaccgag

SEQ ID NO: 25 - DNA, 21 bp, synthetic construct
  source 1..21: mol_type = other DNA; note = S4 DBCO
  modified_base 21: mod_base = OTHER; note = Dibenzylcyclooctyne
ttgatcgacagcagtcagagg

SEQ ID NO: 26 - DNA, 78 bp, synthetic construct
  source 1..78: mol_type = other DNA; note = S1 modified Donor
  modified_base 78: mod_base = OTHER; note = 5(6)-Carboxyfluorescein
ggcactttttcatggcttagcatgtggccaatttttccagtggctcaactagctgtctttttgtcagtctcctgggaa

SEQ ID NO: 27 - DNA, 21 bp, synthetic construct
  source 1..21: mol_type = other DNA; note = S2 Acceptor
  modified_base 21: mod_base = OTHER; note = 5(6)-Carboxytetramethylrhodamine
acccttccgtggtaccgaatc

SEQ ID NO: 28 - DNA, 30 bp, synthetic construct
  source 1..30: mol_type = other DNA; note = TriCross strand01
gtactgtgggctgctctctcagagctgtat

SEQ ID NO: 29 - DNA, 30 bp, synthetic construct
  source 1..30: mol_type = other DNA; note = TriCross strand02
tgcacgcggagacgaagcagcccacagtac

SEQ ID NO: 30 - DNA, 30 bp, synthetic construct
  source 1..30: mol_type = other DNA; note = TriCross strand03
atacagctctgagagtcgtctccgcgtgca

SEQ ID NO: 31 - protein, 570 aa, synthetic construct
  source 1..570: note = IL1RAcP
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

(19) United States
(12) Patent Application Publication
(10) Pub. No.: US 2024/0084044 A1
(43) Pub. Date: Mar. 14, 2024

(54) Heteromultivalent Nucleic Acid Scaffolds and Uses Thereof

(71) Applicant: George Mason University, Fairfax, VA (US)

(72) Inventors: Amanda Haymond Still, Manassas, VA (US); Remi Veneziano, McLean, VA (US); Jessica A. Roman, Fairfax, VA (US); Lance Liotta, Bethesda, MD (US); Alessandra Luchini Kunkel, Burke, VA (US)

(21) Appl. No.: 18/461,041

(22) Filed: Sep. 5, 2023

Related U.S. Application Data
(60) Provisional application No. 63/403,897, filed on Sep. 6, 2022.

(51) Int. Cl. C07K 19/00; C07K 14/54; C07K 14/545; C12N 15/11

(52) U.S. Cl. CPC C07K 19/00; C07K 14/54; C07K 14/545; C12N 15/11; A61K 38/00; C07K 2318/20; C12N 2310/18; C12N 2310/315; C12N 2310/3513

(57) ABSTRACT

The present disclosure relates to heteromultivalent nucleic acid scaffolds and peptides, and to compositions comprising the scaffolds and peptides, and to methods for preparing and using the scaffolds and peptides. The present disclosure also relates to use of the scaffolds, peptides, and compositions for preventing or disrupting interactions between molecules, in particular protein-protein interactions, in particular for the treatment or prevention of disease, in particular cancer, such as breast cancer.

### CROSS-REFERENCE TO RELATED APPLICATIONS

The present application claims priority under 35 U.S.C. § 119(e) to U.S. Provisional Application No. 63/403,897, filed Sep. 6, 2022, which is incorporated herein by reference in its entirety.

### STATEMENT REGARDING FEDERALLY SPONSORED RESEARCH

This invention was made with government support under grant number CA251015 awarded by the National Institutes of Health. The government has certain rights to this invention.

### REFERENCE TO AN ELECTRONIC SEQUENCE LISTING

The present application contains a Sequence Listing which has been submitted in XML format and is herein incorporated by reference in its entirety. Said XML copy, created on Sep. 5, 2023, is named 387351_7003US1_SequenceListing.xml and is 40,752 bytes in size.

### BACKGROUND

Immunotherapies are prevalent treatment options, however, they face a number of challenges. For example, monoclonal antibodies (mAbs) are a popular therapeutic drug for the treatment of a variety of diseases from cancers to autoimmune diseases. mAbs can be designed to be extremely specific to their targets, and they have demonstrated tremendous therapeutic value since their initial use in 1997. Despite their successes, however, mAbs do have several drawbacks. First, they are expensive to produce, and maintaining consistency between production lots can be difficult. Second, mAbs tend to be very large (150 kDa) molecules, which can present drug delivery challenges. Finally, mAbs only select for a single epitope, and given their large size, any attempt to achieve multivalency by combining multiple mAbs would result in a molecule far too large to represent any practical medicinal value.

Single domain antibodies (nanobodies) have been presented as a possible solution for the limitations of mAbs as they are 1/10 the size of antibodies (˜12-15 kDa), are specific, and are easier to modify due to simpler nature of the polypeptide constructs. Achieving multivalency by tethering nanobodies with a disulfide bond at their Fc regions, for example, has been proposed as means to achieve multivalency. Despite these advantages, however, nanobodies share some of the similar drawbacks as mAbs in that they are expensive and complicated to produce. Production of nanobodies requires the use of camelid species, which are larger and thus more costly to house and feed than rodent species and hybridomas used to produce mAbs. Husbandry is also more complex and requires interaction with larger animals, which can be dangerous and require that personnel have special training for interacting with these animals safely. Of note, despite their smaller size, nanobodies and nanobody dimers are still large, which may still provide challenges for drug delivery and possibly be immunogenic.

Mammary cancers have demonstrated remarkable response to immunotherapies; however, frequency of successful treatment remains extremely low at no greater than 40%, and it also remains unpredictable as to which patients will respond to a given immunotherapy. A growing body of evidence suggests that the tumor microenvironment (TME), defined as the extracellular milieu within the architecture of a solid tumor that contains various immune cells, signaling molecules, fibroblasts, and vascular tissue, plays a significant role in promoting a molecular signaling context that is hostile to anti-tumor immunological phenomena and allows for tumor immune evasion, tolerance, and ultimately, disease progression.

As such, there remains a need in the field for new treatment options, such as those that overcome the noted examples of problems with antibody and/or nanobody-based therapeutics, in particular for treating diseases such as cancer, e.g., breast cancer.

### BRIEF SUMMARY

In one aspect, the present invention provides a heteromultivalent nucleic acid scaffold comprising a scaffold strand and at least two staple strands. Each staple strand comprises a peptide attached thereto, and each peptide specifically contacts a different region of a protein, thereby binding the multivalent nucleic acid scaffold to the protein.

In another aspect, the present invention provides a composition comprising a heteromultivalent nucleic acid scaffold; and a pharmaceutically acceptable carrier. The heteromultivalent nucleic acid scaffold comprises a scaffold strand and at least two staple strands. Each staple strand comprises a peptide attached thereto, and each peptide specifically contacts a different region of a protein, thereby binding the multivalent nucleic acid scaffold to the protein.

In some aspects, the present invention provides a method for treating a disease in a subject in need thereof. The method comprises administering a therapeutically effective amount of a composition comprising a heteromultivalent nucleic acid scaffold; and a pharmaceutically acceptable carrier. The heteromultivalent nucleic acid scaffold comprises a scaffold strand and at least two staple strands. Each staple strand comprises a peptide attached thereto, and each peptide specifically contacts a different region of a protein, thereby binding the multivalent nucleic acid scaffold to the protein.

In other aspects, the present invention provides a method for preventing or disrupting association of IL-1RAcP with an ST2/IL3 complex at a surface of a cell. The method comprises contacting IL-1RAcP with a composition comprising a heteromultivalent nucleic acid scaffold. The heteromultivalent nucleic acid scaffold comprises a scaffold strand and at least two staple strands. Each staple strand comprises a peptide attached thereto, and each peptide specifically contacts a different region of a protein, thereby binding the multivalent nucleic acid scaffold to the protein.

In one aspect, the present invention provides a peptide comprising the amino acid sequence set forth in any one of SEQ ID NOs: 1-18.

In another aspect, the present invention provides a polynucleotide comprising the sequence as set forth in any one of SEQ ID NOs: 19-30.

### BRIEF DESCRIPTION OF THE DRAWINGS

The foregoing and other features and advantages of the present invention will be more fully understood from the following detailed description of illustrative embodiments taken in conjunction with the accompanying drawings. It should be understood, however, that the invention is not limited to the precise arrangements and instrumentalities of the embodiments shown in the drawings.

FIGS. 1A-1N: Design of Interfering Peptides based on IL1RAcP hotspots of interaction with ST2/IL-33 binary complex: (A) Model of the extracellular portions of IL-1RAcP, ST2, and IL-33 in their ternary complex. Reported KD for ST2 to IL-33 is 26 pM32. IL-33 Model was rendered with Chimera X and based on the solved ternary complex of IL-1β/IL-1RI with IL-1RAcP. (B) Measured distances between the four individual hotspots and the length of the IL-1RAcP interface with the IL-33/ST2 binary complex. (C) Map of small inhibitory peptide location, based on the above hotspots and the surrounding residues, derived from the wild-type human IL-1RAcP sequence. Peptides 1.7, 2.5, and 3.7 are indicated in lime green, cyan, and navy, respectively. Hotspots are indicated in red. (D) MS spectra, aa sequences, and rendering of small inhibitory peptide 1.4 along with its affinity for the ST2/IL-33 binary complex. (E) MS spectra, aa sequences, and rendering of small inhibitory peptide 2.4 along with its affinity for the ST2/IL-33 binary complex. (F) MS spectra, aa sequences, and rendering of small inhibitory peptide 3.4 along with its affinity for the ST2/IL-33 binary complex. Ten (10) μM of peptide alone or in various combinations were incubated with IL-33, ST2, and IL1RAcP. Inhibition was normalized based on 3 experiments. (G) Linear WT peptides, based on IL1RAcP canonical sequence. (H) Scrambled peptides (I) Alanine mutations, where hotspots were substituted for alanine. (J) Thioether cyclized peptides (K) 100 μM of WT, scrambled, or Thioether cyclized peptides were incubated with HEKBlue IL-33 reporter cells stimulated with 100 pg/mL of IL-33 for 6 hours at 37 C. (L) Sequences of inhibitory peptides. (M) BLAST alignment of complimentary staple strands with 141 nt template, indicating specificity for designated regions. (N) Halotolerance of 3WJ in 1 PBS and 1% DMSO experimental buffer at 4 C. Time designated in hours.

FIGS. 2A-2H: Synthesis of DNA Scaffold and final MSTOP Molecule indicate correct folding, high purity, and structural stability: (A) Schematic of scaffold synthesis 3WJs. Scaffold was designed with Tiamat software. “Arms” length and distances were measured to encompass the full area of the IL-1RAcP interface. (B) Agarose gel demonstrating that 3WJ scaffolding structure folded accurately and efficiently. The fully folded structure (F) of 141 nt resolved at approximately 75 bp, where the template alone (T; 78 nt) resolved just below 50 bp and the three 21 nt staples resolved at about 10 bp. Limited unreacted staple and template strands are observed in the final product. (C) Melting curve indicates 3WJ was heat-stable to 68 C as determined by SYBRgreen fluorescence. (D) FRET analysis of 3WJ in serum indicates stability (E) Schematic of MSTOP synthesis utilizing DBCO-labelled staple strands that are initially reacted with azidolysine modified interfering peptides prior to synthesis of full structure (F) Click chemistry reaction of DBCO-labelled staple strands in the presence of 10-fold excess azidolysine-modified peptides shows complete reaction of the staple strand © in either water (W) or HEPES pH 7.5 buffer (H). Azidolysine-modified peptide 1 was reacted with DBCO-staple 1, etc. (G) MSTOP folding compared to 3WJ without peptides shows no scaffold without peptides remains in the final MSTOP preparation. (H) DLS of MSTOP molecule confirms the expected hydrodynamic radius of the folded molecule.

FIGS. 3A-3C: MSTOP validation showed that the final MSTOP molecule had increased avidity for the ST2/IL-33 binary complex over the parent IL-1RacP: (A) SPR data of IL-1RacP (left) vs. MSTOP (right) for IL-33/ST2 demonstrates improved KD of MSTOP versus parent IL-1RacP. (B) Cross-linking western blot with MSTOP dose curve from 10 μM to 1 nM demonstrates in-vitro efficacy for reducing IL-1RACP/ST2/IL-33 ternary complex formation with recombinant IL-33, ST2, and IL-1RacP. (C) Cross-linking western blot data normalized across three experiments. Trimer-associated IL-1RacP band density was normalized to the total IL-1RacP band density in the lane, inclusive of unbound IL-1RacP at 50 kDa.

FIGS. 4A-4F: MSTOP inhibits IL-33 signaling activation, but not IL-1β signaling activation in a reporter cell model. Whether MSTOP could inhibit IL-33 signaling effectively and specifically in a cell model was determined using the reporter gene cell line, HEK-Blue. (A)

The mechanism for IL-33 activation, showing stable transfection of ST2 with TNFα and IL-1β receptors repressed to prevent nonspecific SEAP expression. (B) IL-33 reporter cells (dark grey) and IL-1β reporter cells (light grey) were used to determine potency and specificity of MSTOP inhibition. HEK-blue cells secrete SEAP as a reporter gene when the specific pathway is activated, which metabolized QunatiBlue substrate to produce a colorimetric product. MSTOP dose curve (10 μM to 10 nM) was compared to treatment with cyclized peptides at 10 μM each (WT). Significant inhibition of IL-33 cells, as compared to IL-1B cells, is observed at every MSTOP concentration. (C) HEK-Blue IL-1β mechanism for SEAP reporter gene expression. (D) QUANTIBlue is metabolized by secreted SEAP, resulting in a colorimetric change that can be quantified by absorbance at 620 nm. (E) IL-33 dose curve demonstrating the metabolism of QUANTI Blue substrate by SEAP expressed due to IL-33 signaling activation. (F) Cellular cytotoxicity assay demonstrating that peptides and MSTOP are not toxic to cells. Concentrations of peptides and MSTOP are in μM.

FIG. 5: schematic representation of an overlay model of the human interleukin-33/suppression of tumorigenicity (ST2)/interleukin-1 receptor accessory protein (IL-33/ST2/IL1RAcP) ternary complex activating MyD88 dependent protein cascades.

FIG. 6: schematic representation of the heteromultivalent nucleic acid scaffold construct that targets the identified hotspot residues.

FIG. 7: schematic representation of IL-33 that has been generated by multiple cell types in a tumor microenvironment regulating the local accumulation of function of Myeloid Derived Suppressor Cells (MDSCs).

### DETAILED DESCRIPTION

### Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics, protein and nucleic acid chemistry, and nucleic acid hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

That the disclosure may be more readily understood, select terms are defined below.

As used herein, the terms “a,” “an,” or “the” are used to include one or more than one unless the context clearly dictates otherwise. By way of example, “an element” means one element or more than one element. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various embodiments of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 and so forth, as well as individual numbers within that range, for example, 1, 2, 2.7, 3, 4, 5, 5.3, and 6. This applies regardless of the breadth of the range.

The terms “comprising,” “comprise,” “comprises,” “comprised,” “have,” “has,” “having,” “include,” “includes,” and “including” in reference to any composition of matter and method described herein is open-ended, and refers to respective component(s)/step(s) thereof, as essential to the invention, yet open to the inclusion of unspecified elements, essential or not. The term “consisting of” in reference to any composition of matter and method described herein, refers to respective component(s)/step(s) thereof, as essential to the invention, yet exclusive of any element not recited with respect to that composition, element, component or method. The term “consisting essentially of” refers to respective component(s)/step(s) thereof, as essential to the invention, yet other elements that can be included in the description of the composition of matter, method or respective component thereof are limited to those that do not materially affect the basic and novel characteristic(s) of the composition of matter, method or respective component thereof.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The terms “effective amount” or “therapeutically effective amount,” as used herein, refer to a sufficient amount of a composition of matter being administered to a subject that will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the heteromultivalent nucleic acid scaffold disclosed herein required to provide a clinically significant decrease in disease symptoms. An appropriate “effective” amount in any individual case is determined using any suitable technique, such as a dose escalation study.

As used herein, the term “nucleic acid scaffold” generally refers to two or more single oligonucleotide strands (staple strands) (e.g., DNA) used to direct the folding of a long, single strand of polynucleotide (scaffold strand) into desired shapes on the order of about 10-nm to a micron or more, and the structures that form therefrom. In some embodiments, the nucleic acid scaffold is a DNA scaffold. In some embodiments, the nucleic acid scaffold is an RNA scaffold. In some embodiments, the nucleic acid scaffold comprises DNA and/or RNA. In some embodiments, the nucleic acid scaffold comprises peptide nucleic acids (PNA) or phosphorothioate-polymerized nucleotides. In some embodiments, at least one peptide is attached to the nucleic acid scaffold. In some embodiments, each staple strand of a nucleic acid scaffold comprises a peptide attached thereto. In some embodiments, a peptide is attached to a staple strand of the nucleic acid scaffold.

As used herein, the term “staple strand(s)” or “helper strand(s)” are used interchangeably and generally refer to oligonucleotides that hold the scaffold DNA in its three-dimensional wireframe geometry. Additional nucleotides can be added to the staple strand at either 5′ end or 3′ end, and those are referred to as “staple overhangs.” Staple overhangs can be functionalized to have desired properties such as a specific sequence to hybridize to a complementary nucleic acid sequence. In some instances, the staple overhang is biotinylated for capturing the DNA nanostructure on a streptavidin-coated bead. In some instances, the staple overhang can be also modified with chemical moieties. Non-limiting examples include Click-chemistry groups (e.g., azide group, alkyne group, DIBO/DBCO), amine groups, and Thiol groups. In some embodiments, some bases located inside the oligonucleotide can be modified using base analogs (e.g., 2-Aminopurine, Locked nucleic acids, such as those modified with an extra bridge connecting the 2′ oxygen and 4′ carbon) to serve as linker to attach functional moieties (e.g., lipids, proteins). Alternatively DNA-binding proteins or guide RNAs can be used to attach secondary molecules to the DNA scaffold.

As used herein, the terms “inhibit(ing),” “prevent(ing),” “block(ing),” and “disrupt(ing)” are used interchangeably and generally refer to the ability of a compound e.g., nucleic scaffold as described herein, e.g., a nucleic acid scaffold comprising a peptide attached thereto as described herein, to reduce or impede a described function, such as inhibiting a targeted protein-protein interaction, e.g., inhibiting IL1RAcP coupling to ST-2/IL33. Preferably, inhibition, prevention, or disruption is by at least 10%, more preferably by at least 25%, even more preferably by at least 50%, and most preferably, the function is inhibited by at least 75%.

As used herein, the term “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, the terms “conservative variation” or “conservative substitution” generally refers to the replacement of an amino acid residue by a different, biologically similar residue. Conservative variations or conservative substitutions are not likely to change the shape of the peptide chain. Examples of conservative variations, or conservative substitutions, include, for example, the replacement of one hydrophobic residue such as isoleucine, valine, leucine or methionine for a different hydrophobic residue, or the substitution of one polar residue for a different polar residue, such as the substitution of arginine for lysine, glutamic for aspartic acid, or glutamine for asparagine.

As used herein, the term “isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the invention. Molecules may be modified in many ways, including chemically, structurally, and functionally. Cells may be modified through the introduction of nucleic acids.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

### Nucleic Acid Scaffolds

In one aspect, the present invention provides a heteromultivalent nucleic acid scaffold comprising a scaffold strand and at least two staple strands, wherein each staple strand comprises a peptide attached thereto, and wherein each peptide specifically contacts a different region of a protein, thereby binding the heteromultivalent nucleic acid scaffold to the protein.

While not wishing to be bound by theory, in some embodiments, the principles of nucleic acid origami (e.g., DNA origami) technique can be used to aide in the synthesis of nucleic acid scaffolds. Briefly, in various embodiments, the DNA origami technique enables high fidelity folding (>95%) of a single stranded DNA scaffold into nanoparticles (DNA-NPs) of any arbitrary shape and size using multiple short oligonucleotides called “staple” strands that hybridize to specific regions of the scaffold strand via Watson Crick base pairing. This sequence specificity makes nucleic acid origami highly programmable and allow for orthogonal conjugation of organic and inorganic molecules with nanoscale precision. Methods useful in the making of nucleic acid origami structures can be found, for example, in U.S. Pat. Nos. 7,842,793 and 9,340,416; U.S. Pat. App. Pub. Nos. 2008/0287668 and 2010/0216978; Rothemund, P. W., “Folding DNA to create nanoscale shapes and patterns,” Nature 440:297-302 (2006); Douglas et al., “Self-assembly of DNA into nanoscale three-dimensional shapes,” Nature 459:414-418 (2009); Dietz et al., “Folding DNA into twisted and curved nanoscale shapes,” Science 325:725-730 (2009); and CADnano software, available at cadnano.org; each of which is incorporated by reference in its entirety.

In some embodiments, the heteromultivalent nucleic acid scaffold structure incorporates nucleic acid (e.g., DNA) as a building material to make nanoscale shapes. The structures can be manipulated to fold into a variety of 2D and 3D shapes, e.g., in some embodiments, depending on the design and location of the staple strands. For example, in one embodiment, the process involves the folding of one or more “scaffold” strands (e.g., scaffold DNA strands) into a particular shape using a plurality of rationally designed “staple” strands (e.g. staple DNA strands). In some embodiment, the sequences of the staple strands are designed such that they hybridize to particular portions of the scaffold strands and, in doing so, force the scaffold strands into a particular shape.

In other embodiments, the heteromultivalent nucleic acid scaffold is a multi-arm scaffold comprising peptide(s) (including, but not limited to, peptide(s) that inhibit protein-protein interaction(s)) tethered thereto that keeps them in proximity to each other. In various embodiments, the result is a heteromultivalent molecule that can target one or more interacting hotspot residues on a protein of interest, thereby spanning the entire coupling interface of the protein of interest with another protein or protein complex, but is small and flexible enough to conform to the complex protein surface topology.

In one embodiment, the scaffold strand and/or the at least two staple standards, independently, each comprises DNA and/or RNA.

In other embodiments, the scaffold strand and/or the at least two staples strands, independently, each comprises single-stranded nucleic acid. In one embodiment, the scaffold strand and/or the at least two staples strands, independently, each comprises single-stranded DNA (ssDNA).

In another embodiment, the scaffold strand and/or the at least two staples strands, independently, each is PEGylated.

In some embodiments, the scaffold strand and/or the at least two staples strands, independently, each comprises peptide nucleic acids (PNA) or phosphorothioate-polymerized nucleotides.

In other embodiments, the nucleic acid scaffold is modified. In one embodiment, the nucleic acid scaffold is modified with one or more hexaethylene glycol groups. In another embodiment, modification with one or more hexaethylene glycol groups enhances stability of scaffold up to 60 hours relative to a similar scaffold having less or no hexaethylene glycol groups. In some embodiments, the backbone of the scaffold is modified by introducing phosphorothioate bonds in amounts sufficient to prevent degradation of the scaffold by nucleases.

In one embodiment, each of the nucleic acid strands of the heteromultivalent nucleic acid scaffold is made of a nucleic acid selected from the group comprising DNA, RNA, locked nucleic acid (LNA), bridged nucleic acid (BNA), PNA, or any nucleic acids from the class of XNA (xeno nucleic acid-a class of nucleic acids with an unnatural moiety replacing the sugar molecule). See e.g., Arun Richard Chandrasekaran, “Nuclease resistance of DNA nanostructures,” Nature Reviews Chemistry, volume 5, pages 225-239 (2021), which is incorporated herein by reference in its entirety. The class of XNAs comprises, for example, CeNA, ANA, FANA, TNA, HNA, LNA, GNA and PNA and the binding affinities of many of them are described for example in e.g., Pinheiro, V. B., et al. (Synthetic Genetic Polymers Capable of Heredity and Evolution. (2012) Science, 336, 341-344). In one embodiment, each of the strands is made of a different nucleic acid. In another embodiment, at least one strand comprises several different types of nucleic acids. The stability of a natural nucleic acid hybrid can for example be increased by adding non-natural nucleotides such as LNA, BNA, and PNA. In one embodiment, at least one strand of the heteromultivalent nucleic acid scaffold comprises at least 2% of a non-natural nucleotide, more preferably at least 5%, even more preferably at least 10%, yet more preferably at least 15% and most preferably at least 20%. These non-natural nucleotides can all be located next to each other in a strand or interspersed among natural nucleotides.

In other embodiments, each strand is made of one nucleic acid over its entire length. In another embodiment, at least two, three, four, five or most preferably all of the nucleic acid strands of the heteromultivalent nucleic acid scaffold are made of the same nucleic acid.

In some embodiments, staple strands of the heteromultivalent nucleic acid scaffold, independently, each comprises a length of at least 5 nucleotides, illustratively, about 5 to about 10,000 nucleotides, about 10 to about 7,500 nucleotides, about 100 to about 5,000 nucleotides, about 500 to about 2,500, about 1,000 to about 1,500 nucleotides in length. In one embodiment, staple strands of the nucleic acid scaffold, independently, each comprises a length of at least 5, 6, 7, 8, 9, 10, 11, 12, 13, 14, 15, 16, 17, 18, 19, 20, 25, 30, 35, 40, 50, 60, 70, 80, 90, 100, 110, 120, 130, 140, 150, 160, 170, 180, 190, 200, 300, 400, 500, 600, 700, 800, 900, 1,000, 1,500, 2,000, 3,000, 4,000, 5,000, or 10,000 nucleotides. In another embodiment, staple strands of the nucleic acid scaffold, independently, each comprises a length of no more than 5, 10, 11, 12, 13, 14, 15, 16, 17, 18, 19, 20, 25, 30, 35, 40, 50, 60, 70, 80, 90, 100, 110, 120, 130, 140, 150, 160, 170, 180, 190, 200, 300, 400, 500, 600, 700, 800, 900, 1,000, 1,500, 2,000, 3,000, 4,000, 5,000, or 10,000 nucleotides.

In other embodiment, scaffold strand(s) of the heteromultivalent nucleic acid scaffold, independently, each comprises a length of at least 5 nucleotides, illustratively, about 5 to about 10,000 nucleotides, about 10 to about 7,500 nucleotides, about 100 to about 5,000 nucleotides, about 500 to about 2,500, about 1,000 to about 1,500 nucleotides in length. In one embodiment, scaffold strand(s) of the nucleic acid scaffold, independently, each comprises a length of at least 5, 6, 7, 8, 9, 10, 11, 12, 13, 14, 15, 16, 17, 18, 19, 20, 25, 30, 35, 40, 50, 60, 70, 80, 90, 100, 110, 120, 130, 140, 150, 160, 170, 180, 190, 200, 300, 400, 500, 600, 700, 800, 900, 1,000, 1,500, 2,000, 3,000, 4,000, 5,000, or 10,000 nucleotides. In another embodiment, scaffold strand(s) of the nucleic acid scaffold, independently, each comprises a length of no more than 5, 6, 7, 8, 9, 10, 11, 12, 13, 14, 15, 16, 17, 18, 19, 20, 25, 30, 35, 40, 50, 60, 70, 80, 90, 100, 110, 120, 130, 140, 150, 160, 170, 180, 190, 200, 300, 400, 500, 600, 700, 800, 900, 1,000, 1,500, 2,000, 3,000, 4,000, 5,000, or 10,000 nucleotides.

In some embodiments, the heteromultivalent nucleic acid scaffold comprises one or more sequences as set forth in any one of SEQ ID NOs: 19-30 (Table 1).

TABLE 1
Nucleic Acids

| SEQ | | |
|---|---|---|
| ID | | |
| NO | Description | Sequence (5' to 3') |
| 19 | S1 (Scaffold) | TGGGAAGGCACTTTTTCATGGCTTAG |
| | | CATGTGGCCAATTTTTCCAGTGGCTC |
| | | AACTAGCTGTCTTTTTGTCAGTCTCC |
| 21 | S3 (Staple2) | GTACACCGGTTGGTCACCGAG |
| 23 | S2-DBCO | ACCCTTCCGTGGTACCGAATC-DBCO |
| 24 | S3-DBCO | GTACACCGGTTGGTCACCGAG-DBCO |
| 25 | S4-DBCO | TTGATCGACAGCAGTCAGAGG-DBCO |
| 26 | S1-modified | GGCACTTTTTCATGGCTTAGCATGTG |
| | Donor | GCCAATTTTTCCAGTGGCTCAACTAG |
| | | CTGTCTTTTTGTCAGTCTCCTGGGA |
| | | A-FAM |
| 27 | S2-Acceptor | ACCCTTCCGTGGTACCGAATC-TAM |
| 28 | TriCross | GTACTGTGGGCTGCTCTCTCAGAGC |
| | strand01 | TGTAT |
| 29 | TriCross | TGCACGCGGAGACGAAGCAGCCCAC |
| | strand02 | AGTAC |
| 30 | TriCross | ATACAGCTCTGAGAGTCGTCTCCGC |
| | strand03 | GTGCA |

In some embodiments, the heteromultivalent nucleic acid scaffold comprises one or more sequences comprising a tetrazine-reactive moiety and/or an amine-reactive functional group. In one embodiment, the tetrazine-reactive moiety is dibenzocyclooctyne (DBCO), trans-cyclooctene (TCO), difluorinated cyclooctyne (DIFO), bicyclononyne (BCN), or dibenzocyclooctyne (DICO). In another embodiment, the amine-reactive functional group is an isothiocyanate, isocyanate, acyl azide, N-hydroxysuccinimide (NHS) ester, sulfonyl chloride, aldehyde, glyoxal, epoxide, carbonate, arylating agent, imidoester, or carbodimide.

In still further embodiments, the heteromultivalent nucleic acid scaffold comprises one or more sequences comprising 5(6)-carboxyfluorescein (FAM) and/or 5(6)-Carboxytetramethylrhodamine (TAMRA) at a 5′ and/or 3′ end of a strand.

In other embodiments, the heteromultivalent nucleic acid scaffold is non-toxic to a subject administered the scaffold.

In some embodiments, the heteromultivalent nucleic scaffold comprises one or more attachment sites for one or more peptides.

In some embodiments, protein painting techniques can be utilized to aide in the design of the peptide(s) to be attached to the heteromultivalent nucleic acid scaffold. See, for example, Luchini, A., et al., Nat Commun 5, 4413 (2014), and U.S. Pat. No. 10,126,304, the disclosures of both of which are incorporated herein by reference in their entireties.

In some embodiments, the heteromultivalent scaffold comprises more than one peptide attachment site, and each of the attachment sites are arranged such that the spacing and orientation of the attachment sites allows for the targeting of more one hotpot in a given protein-protein interaction. See, for example, FIG. 6, which is a schematic of a DNA scaffold comprising attachments sites spaced and oriented in such a way so as to allow for the targeting of four different hotspots of the IL1RAcP interface with the IL-33/ST2 complex in accordance with one embodiment. In some embodiments, a heteromultivalent nucleic acid scaffold comprising at least one attached peptide is stable for at least 2 hours, as, for instance, can be measured via FRET and gel electrophoresis. In some embodiments, use of a heteromultivalent nucleic acid scaffold comprising at least one attached peptide to inhibit a protein-protein interaction results in a 10-fold decrease in K_{d} value as compared to a control inhibiting peptide. In some embodiments, use of a nucleic acid scaffold comprising at least one attached peptide to inhibit a protein-protein interaction results a 10-fold decrease in IC50 as compared to equimolar treatment with a control inhibiting peptide. In some embodiments, the molecular weight of a heteromultivalent nucleic acid scaffold comprising at least one attached peptide is about 1 kDa to about 10 MDa, about 2 kDa to about 9 MDa, or about 3 kDa to about 8 MDa. In some embodiments, the molecular weight of a heteromultivalent nucleic acid scaffold comprising at least one attached peptide is about 1 MDa or less. In some embodiments, the molecular weight of a heteromultivalent nucleic acid scaffold comprising at least one attached peptide is 50 kDa or less.

In one embodiment, the peptide is attached to the at least two staple strands, independently, each internally or at their 5′- or 3′-end.

In another embodiment, at least one staple strand comprises a small molecule.

In one embodiment, the small molecule is a compound having a molecular weight equal to or less than about 5,000, 4,000, 3,000, 2,000, 1,000, 900, 800, 700, 600, 500, 400, 300, or 200 grams per mole.

In some embodiments, the small molecule is not: (i) a peptide or (ii)an otherwise proteinaceous compound.

In other embodiments, the small molecule provides specificity to target e.g., a kinase. In one embodiment, the small molecule is a kinase inhibitor for directly blocking function of a kinase. In another embodiment, one strand of the heteromultivalent nucleic acid scaffold is attached to a small molecule that fits into a binding pocket on the targeted protein, such as a cofactor or substrate binding pocket on an enzyme, or a metal binding pocket.

In some embodiments, the small molecule comprises one or more of a mitotic kinase inhibitor (or mitotic inhibitor) and/or checkpoint inhibitor. In some embodiments, the mitotic kinase inhibitor is an inhibitor of a polo-like kinase (PLK), an Aurora kinase, cyclin-dependent kinase (CDK)1, CDK2, HASPIN, monopolar spindle 1 kinase (Mps1), a NimA-related kinase (NEK), GSK461364, BI2536, Tak960, NMS-P937, volasertib, Chk 1 Kinase Inhibitor LY2603618, AU14022, YK-4-279, AZ703, alisertib, prexasertib, or AZD7762.

In other embodiments, the small molecule comprises one or more of an immune checkpoint inhibitor comprising a siRNA or inhibitor against one or more of PD-L1, PD-1, TIM-3, LAG-3, or CTLA-4.

In one embodiment, the small molecule is an inorganic compound. In some embodiments, the inorganic compound is a metal cluster.

In other embodiments, the small molecule is an agonist or antagonist.

In another embodiment, the small molecule is a compound selected from the group consisting of amino acid, amino acid analogue, nucleotide, nucleotide analogue, organic compound or inorganic compound (i.e. including a heterorganic compound or organometallic compound), and salts, esters and other pharmaceutically acceptable forms thereof.

In other embodiments, the heteromultivalent nucleic acid scaffold comprises at least one detectable label. A detectable label can be any conjugated molecule that allows the heteromultivalent nucleic acid scaffold that carries the label to be detected. Suitable detectable labels include, but are not limited to, fluorescent moieties, radio-labels, biotin and magnetic particles. A heteromultivalent nucleic acid scaffold that comprises a detectable label is useful for use in diagnosis. The detectable label allows the user to detect whether scaffold is present or absent in the diagnostic assay. In one embodiment, the detectable label is bound to the heteromultivalent nucleic acid scaffold or to a peptide of the scaffold. In another embodiment, the detectable label is an integral part of the peptide (e.g., in the form of a fluorescent amino acid.) In some embodiments, the label is bound to the heteromultivalent nucleic acid scaffold covalently or non-covalently.

In other embodiments, at least one staple strand comprises a polypeptide or protein having a size sufficient to comprise a secondary structure.

In other embodiments, the peptide is attached to the least two staple strands, independently, each by NETS-based coupling via amine groups, maleimide coupling via cysteine residues, carbodiimide coupling via carboxyl groups, copper-free click chemistry, or by complementary nucleic acid overhangs.

In one embodiment, the heteromultivalent nucleic acid scaffold further comprises a covalent bond between two individual nucleic acid strands of the scaffold. In some embodiment, the heteromultivalent nucleic acid scaffold further comprises disulfide bonds (thiol-thiol). In other embodiments, the heteromultivalent nucleic acid scaffold further comprises intra and inter-strand disulfide bonds (thiol-thiol).

In one embodiment, the peptide is attached to the least two staple strands, independently, each by copper-free click chemistry, such as through use of azide-cyclooctine, e.g., DBCO. Click chemistry is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition (e.g., 1,3-dipolar cycloaddition between an azide and an alkyne to form a 1,2,3-triazole linker); thiolyne addition; imine formation; Diels-Alder reactions between tetrazines and trans-cyclooctene (TCO); and Michael additions (e.g., maleimide addition).

Regarding the copper-free click chemistry reactions, click chemistry reactions between alkynes and azides typically require the addition of a copper catalyst to promote the 1,3-cycloaddition reaction, and are known as copper-catalyzed azide-alkyne cycloaddition (CuAAC) reactions. However, click chemistry reactions between cyclooctyne or cyclooctyne derivatives and azides typically do not require the addition of a copper catalyst, and instead proceed via strain-promoted azide-alkyne cycloaddition (SPAAC) (Debets, M. F., et al., Bioconjugation with strained alkenes and alkynes. Acc Chem Res, 2011. 44(9): p. 805-15).

In some embodiments, the peptide is a cyclized peptide. In other embodiments, cyclized peptides are provided that may be more stable in solution. Various methods for cyclizing peptides, e.g., via disulfide bond formation, are known in the art. In other embodiments, the cyclized peptide is a thioether cyclized peptide. In one embodiment, the cyclized peptide comprises the amino acid sequence as set forth in any one of SEQ ID NOs: 10-12 and 16-18. In another embodiment, the peptide is attached to at least one molecule, such as a therapeutic molecule.

In other embodiments, the peptides comprise modifications for stability of the peptides against protease activity or to rigidify structure to reduce entropic costs of binding. In some embodiment, the peptides comprise D-amino acids, peptide bond isosteres, and/or N-methylated peptide bonds.

In another embodiment, different peptides are attached to each of the at least two staple strands. In some embodiments, each staple strand comprises a single peptide attached thereto, wherein each peptide of the nucleic acid scaffold has a unique amino acid sequence.

In some embodiments, the peptide is capable of preventing or inhibiting protein-protein interaction. In some embodiments, the peptide is capable of binding to a target. In other embodiments, the peptide is capable of binding to a protein, such as a receptor. In some embodiments, the peptide that binds to a receptor is capable of increasing or decreasing the function of the receptor.

In other embodiments, the heteromultivalent nucleic acid scaffold comprises peptides that can be used for delivery or targeting purposes. For example, in some embodiment, blood-brain barrier penetrating peptides, cell-penetrating peptides (CPP), or nuclear localization sequences are attached to one or more strands of the scaffold to guide the scaffold either into a cell or to a particular cellular compartment. In other embodiments, ligands for receptors on certain cell types are added to localize the scaffold to a given cell type.

The present disclosure embraces compositions and methods for revealing binding sites between proteins, proteins and nucleic acids, or proteins and small molecules, including use of protein painting techniques, for example, as disclosed in U.S. Pat. No. 10,126,304; U.S. Pat. App. Pub. No. 2022/0064259; Luchini, A., et al., Nat Commun 5, 4413 (2014); each of which is incorporated by reference in its entirety. In some embodiment, protein painting exposes and sequences hidden native hot spot domains of protein-protein interactions and, based thereon, one or more peptides that target the interaction sites (e.g., so called “hot spots”) can be prepared for use in the scaffolds, compositions, and methods disclosed herein.

For example, in some embodiments, paint molecules (e.g., as disclosed in U.S. Pat. No. 10,126,304; U.S. Pat. App. Pub. No. 2022/0064259; and/or Luchini, A., et al., Nat Commun 5, 4413 (2014)) coat the surface of native protein complexes but cannot gain access to solvent-inaccessible protein-protein interface domains. For example, in one embodiment, each molecular paint spans less than about 5, 4, or 3 amino acids. In another embodiment, cleavage (e.g., trypsin cleavage) is blocked by presence of paint molecules, which bind non-covalently near cleavage (e.g., trypsin) consensus sequences. Following dissociation of painted proteins the area of interaction remains unpainted and is susceptible to cleavage.

In other embodiments, proteins are pulsed with molar (e.g., 10 molar) excess small molecule molecular paints; then unbound paint molecules are washed away e.g., with gel filtration chromatography (e.g., Sephadex G-25); then protein complex is dissociated and denatured e.g., with 2 M urea; then proteins are linearized e.g., by dithiothreitol (DTT) reduction and iodoacetamide alkylation; then linearized proteins are subjected to trypsin digestion; then tryptic fragments are analyzed e.g., by reversed-phase liquid chromatography nanospray tandem mass spectrometry (LC-MS/MS).

Thus, in some embodiment, by employing small organic molecule masking pigments that bind to the surface of proteins, for example, the small organic molecules can be used to “paint” the exposed regions of a protein in solution for direct positive identification and sequencing of the contact interface region between two interacting native unmodified proteins, for instance.

In other embodiments, the peptide targets a protein-protein interaction hotspot. For instance, the peptide can target one or more amino acids of a protein-protein binding interface that contribute to the coupling stability and free energy of a binding site of a given protein-protein interaction. For example, in some embodiments, a peptide as described herein can target one or more of R157, K238, K343, and K346 positions of IL1RAcP (SEQ ID NO: 31).

(SEQ ID NO: 31)
MTLLWCVVSLYFYGILQSDASERCDDWGLDTMRQIQVFEDE
PARIKCPLFEHFLKFNYSTAHSAGLTLIWYWTRQDRDLEE
PINFRLPENRISKEKDVLWFRPTLLNDTGNYTCMLRNTTY
CSKVAFPLEVVQKDSCFNSPMKLPVHKLYIEYGIQRITCP
NVDGYFPSSVKPTITWYMGCYKIQNFNNVIPEGMNLSFLI
ALISNNGNYTCVVTYPENGRTFHLTRTLTVKVVGSPKNAV
PPVIHSPNDHVVYEKEPGEELLIPCTVYFSFLMDSRNEVW
WTIDGKKPDDITIDVTINESISHSRTEDETRTQILSIKKV
TSEDLKRSYVCHARSAKGEVAKAAKVKQKVPAPRYTVELA
CGFGATVLLVVILIVVYHVYWLEMVLFYRAHFGTDETILD
GKEYDIYVSYARNAEEEEFVLLTLRGVLENEFGYKLCIFD
RDSLPGGIVTDETLSFIQKSRRLLVVLSPNYVLQGTQALL
ELKAGLENMASRGNINVILVQYKAVKETKVKELKRAKTVL
TVIKWKGEKSKYPQGRFWKQLQVAMPVKKSPRRSSSDEQG
LSYSSLKNV.

Interleukin-1 receptor accessory protein isoform 1 precursor (Homo sapiens) sequences are disclosed by National Center for Biotechnology Information (NCBI) Accession No.: NP_001161400.1, which is incorporated herein by reference in its entirety.

In some embodiments, the nucleic acid scaffold comprises at least two peptides attached thereto, and each of the peptides target at least two different protein-protein interaction hotspots. In some embodiments, an individual peptide targets more than one protein-protein interaction hotspot.

In other embodiments, binding of the heteromultivalent nucleic acid scaffold to the protein prevents or disrupts association of the protein with a binding partner by mimicking at least two different protein-protein interaction hotspots with which the protein interacts with the binding partner. In some embodiments, the protein and/or the binding partner each comprises a protein complex comprising two or more proteins. In some embodiments, the protein and/or the binding partner each comprises a single polypeptide chain. In one embodiment, the protein is an accessory protein. In another embodiment, the accessory protein is IL-1 Receptor Accessory Protein (IL-1RAcP). In other embodiments, the binding partner is a complex comprising a ligand and a receptor. In some embodiments, the ligand is IL-33. In other embodiments, the receptor is ST2. In one embodiment, the protein is IL-1RAcP and the binding partner is a ligand/receptor complex comprising IL-33/ST2.

In one embodiment, at least two peptides are attached to a nucleic acid scaffold. For instance, in some embodiments, 2, 3, 4, 5, 6, 7, 8, 9, 10, 11, 12, 13, 14, 15, 16, 17, 18, 19, or 20 peptides are attached to the nucleic acid scaffold. In other embodiments, two or more peptides are attached to the same nucleic acid strand of the heteromultivalent nucleic acid scaffold. In one embodiment, no more than one peptide is attached to one strand of the heteromultivalent nucleic acid scaffold.

In some embodiments, different peptides are each attached independently to at least two staple strands of the nucleic acid scaffold. In other embodiments, each peptide is independently attached to the nucleic acid scaffold by the same and/or by different means of attachment, such as those described herein. In another embodiment, the peptide comprises the amino acid sequence of any one of SEQ ID NOs: 1-18 (Table 2).

TABLE 2
Peptides

| SEQ | | |
|---|---|---|
| ID | | |
| NO | Description | Sequence |
| 1 | Wild type parent peptide 1.0- | EYGIQRITC |
| | hotspot R157 | |
| 2 | Wild type parent peptide 2.0- | VGSPKNAVPP |
| | hotspot K238 | |
| 3 | Wild type parent peptide 3.0- | GEVAKAAKVK |
| | hotspot K343/K346 | |
| 4 | Alanine Substitution Peptide | EYGIQAITC |
| | 1.1-hotspot R157 | |
| 5 | Alanine Substitution Peptide | VGSPANAVPP |
| | 2.1-hotspot K238 | |
| 6 | Alanine Substitution Peptide | GEVAAAAAVK |
| | 3.1-hotspot K343/K346 | |
| 7 | Scrambled Peptide 1.2- | RGQETCYII |
| | hotspot R157 | |
| 8 | Scrambled Peptide 2.2- | VNPSPVPAGK |
| | hotspot K238 | |
| 9 | Scrambled peptide 3.2-hotspot | KAAEKVAGVK |
| | K343/K346 | |
| 10 | Thioether cyclized peptide 1.3- | EYGIQRITC |
| | first and last residues linked- | |
| | Hotspot R157 | |
| 11 | Thioether cyclized peptide 2.3- | VGSPKNAVPPC |
| | first and last residues linked- | |
| | Hotspot K238 | |
| 12 | Thioether cyclized peptide 3.3- | GEVAKAAKVKC |
| | first and last residues linked- | |
| | Hotspot K343/K346 | |
| 13 | Truncation peptide 1.4- | EYGIRC |
| | Hotspot R157 | |
| 14 | Truncation peptide 2.4- | SPKNV |
| | Hotspot K238 | |
| 15 | Truncation peptide 3.4- | VAKAAKV |
| | Hotspot K343/K346 | |
| 16 | Azidolysine peptide-Hotspot | Ac-C-K(N₃)- |
| | R157 (disulfide link between | EYGIQRITC- |
| | terminal Cys residues) | NH₂ |
| 17 | Azidolysine peptide-Hotspot | Ac-A-K(N₃)-VG |
| | K238 (thioether cycle between | SPKNAVPPC- |
| | terminal Ala and Cys residues) | NH₂ |
| 18 | Azidolysine peptide-Hotspot | Ac-C-K(N₃)-GE |
| | K343/K346 (disulfide bond | VAKAAKVKC- |
| | between terminal Cys residues) | NH₂ |

Absent an express indication of the N-terminus and/or C-terminus of a peptide set forth herein, the peptide is to be read in the N-terminus to C-terminus orientation. In some embodiments, individual residues are indicated by the identity of the amino acid using a standard one- and/or three-letter code known to one of ordinary skill in the art.

In some embodiments, the peptide comprises an amino acid sequence, wherein the peptide further comprises N-terminal acetylation and/or C -terminal amidation and/or azidolysine.

In some embodiments, the peptide comprises the amino acid sequence of any one of SEQ ID NOs: 1-18, wherein the peptide further comprises N-terminal acetylation and/or C-terminal amidation and/or azidolysine. In some embodiments, the peptide comprises the amino acid sequence of any one of SEQ ID NOs: 1-18, e.g., any one of SEQ ID NOs: 10-12 and 16-18, wherein the peptide is cyclized via disulfide-bond formation between an amino- and/or carboxy-terminal cysteine residue. In some embodiments, the peptide comprises the amino acid sequence of any one of SEQ ID NOs: 1-18, e.g., any one of SEQ ID NOs: 10-12 and 16-18, wherein the peptide is cyclized via head-to-tail condensation with the addition of terminal Ala or Gly residues. In some embodiments, the peptide comprises the amino acid sequence of any one of SEQ ID NOs: 1-18, e.g., any one of SEQ ID NOs: 10-12 and 16-18, wherein the peptide is cyclized via amino and/or carboxy terminal cysteine residues using thioether bonds. In some embodiments, the peptide comprises an amino acid sequence of at least about 70%, at least about 75%, at least about 80%, at least about 85%, at least about 90%, at least about 91%, at least about 92%, at least about 93%, at last about 94%, at least about 95%, at least about 96%, at least about 97%, at least about 98%, at least about 99%, or at least about 99.9% identity to the amino acid sequence of any one of SEQ ID NOs: 1-18. In some embodiments, the peptide consists essentially of the amino acid sequence of any one of SEQ ID NOs: 1-18. In some embodiments, the peptide consists of the amino acid sequence of any one of SEQ ID NOs: 1-18.

In some embodiments, the peptide is equal to or less than: 20, 19, 18, 17, 16, 15, 14, 13, 12, 11, 10, 9, 8, 7, 6, 5, 4, or 3 amino acid residues in length. In some embodiments, the peptide is equal to or no more than: 20, 19, 18, 17, 16, 15, 14, 13, 12, 11, 10, 9, 8, 7, 6, 5, 4, or 3 amino acid residues in length. In some embodiments, the peptide is about 3 to about 20, about 4 to about 19, about 5 to about 18, about 6 to about 17, about 7 to about 16, about 8 to about 15, about 9 to about 14, about 10 to about 12 amino acid residues in length. In other embodiments, the peptides provided herein have a length of about 5 amino acids to about 50 amino acids. For example, in some embodiments, a peptide has a length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In another embodiment, a peptide can have a length of, without limitation, about 5 to about 15 amino acids, about 15 to about 20 amino acids, about 20 to about 25 amino acids, about 25 to about 30 amino acids, about 30 to about 35 amino acids, about 35 to about 40 amino acids, about 40 to about 45 amino acids, about 45 to about 50 amino acids, about 10 to about 20 amino acids, about 20 to about 30 amino acids, about 30 to about 40 amino acids, or about 40 to about 50 amino acids.

In some embodiments, the peptides provided herein can include variants e.g., a peptide variant having one or more (e.g., one, two, three, four, five or more) substitutions, insertions, deletions, and/or additions (and combinations thereof) relative to an amino acid sequence disclosed herein.

Amino acid substitutions can be conservative or non-conservative amino acid substitutions. Conservative amino acid substitutions can be, for example, aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids. Conservative amino acid substitutions also include groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Non-conservative amino acid substitutions typically entail exchanging a member of one of the classes described above for a member of another class.

In some embodiment, a C-terminal amide, or other C-terminal capping moiety can be present in peptides described herein. In one embodiment, a peptide described herein is amidated at the C-terminal. In other embodiments,

In some embodiments, the peptide specifically binds or is capable of specifically binding to a target. In some embodiments, the peptide inhibits or is capable of inhibiting a targeted protein-protein interaction. In some embodiments, the peptide comprises a nucleic acid component that specifically hybridizes to a target, such as hybridizing to a complementary target nucleic acid sequence.

In some embodiments, the peptide is capable of preventing or disrupting association of IL1RAcP with an ST2/IL3 complex at a surface of a cell, e.g., an MDSC. In some embodiments, the peptide that inhibits IL1RAcP coupling to IL-33 bound ST2 targets R157, K238, K343, and K346 of IL1RAcP (SEQ ID NO: 31). In some embodiments, the Kd value of the peptide binding to the IL1RAcP hotspot is between about 10⁻⁵ and 10⁻¹² M, between about 10⁻⁶ and 10⁻¹¹ M, between about 10⁻⁷ and 10⁻¹⁰ M, or between about 10⁻⁸ to about 10⁻⁹ M. In some embodiments, the Kd value of the peptide that inhibits IL1RAcP coupling to IL-33 bound ST2 has a Kd value between 10⁻⁸ to 10⁻⁹ M for the IL-33/ST2 complex. In some embodiments, the peptide is capable of inducing an IL-33 signaling reduction of >50% when HEKBlue reporter cells are treated with 1 μM of each peptide.

In other aspects, the present provides a peptide as described herein.

In one embodiment, the peptide comprises the amino acid sequence set forth in any one of SEQ ID NOs: 1-18. In another embodiment, the peptide comprises the amino acid sequence set forth in any one of SEQ ID NOs: 1-18, wherein the peptide comprises N-terminal acetylation and/or C-terminal amidation. In some embodiments, the peptide comprises the amino acid sequence set forth in any one of SEQ ID NOs: 1-18, wherein the peptide comprises N-terminal acetylation. In other embodiments, the peptide comprises the amino acid sequence set forth in any one of SEQ ID NOs: 1-18, wherein the peptide comprises C-terminal amidation. In other embodiments, the peptide comprises the amino acid sequence set forth in any one of SEQ ID NOs: 1-18, wherein the peptide comprises N-terminal acetylation and C-terminal amidation.

### Compositions

In another aspect, the present provides a composition comprising the nucleic acid scaffold as described herein. Among the compositions are pharmaceutical compositions and formulations for administration. Also provided are therapeutic methods for administering the nucleic acid scaffold and compositions to subjects.

In some embodiments, the subject is a human or non-human mammal. In other embodiments, non-human mammals include, but are not limited to, livestock and pets, such as ovine, bovine, porcine, canine, feline and murine mammals, as well as simian and non-human primate mammals. In one embodiment, the subject is human. In another embodiment, the subject is a patient.

As such, in one embodiment, the composition is a pharmaceutical composition comprising the nucleic acid scaffold as described herein and a pharmaceutically acceptable carrier.

Also provided are compositions for administration, including pharmaceutical compositions and formulations, such as unit dose form compositions including the amount of nucleic acid scaffold for administration in a given dose or fraction thereof. In some embodiments, the pharmaceutical compositions and formulations comprise one or more optional pharmaceutically acceptable carrier or excipient. In some embodiments, the composition includes at least one additional therapeutic agent.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some embodiments, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some embodiments are included in the compositions. Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated, where the respective activities do not adversely affect one another. Such active ingredients are suitably present in combination in amounts that are effective for the purpose intended. Thus, in some embodiments, the pharmaceutical composition further includes other pharmaceutically active agents or drugs. The pharmaceutical composition in some embodiments contains the nucleic acid scaffold in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single administration of the nucleic acid scaffold, by multiple administrations of the nucleic acid scaffold, or by continuous administration of the nucleic acid scaffold.

Formulations include those for oral, intravenous, intraperitoneal, subcutaneous, pulmonary, transdermal, intramuscular, intranasal, buccal, sublingual, or suppository administration. In some embodiments, the nucleic acid scaffold is administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some embodiments be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some embodiments be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

### Methods

In another aspect, the present invention provides a method for treating a disease in a subject in need thereof, the method comprising administering a therapeutically effective amount of a composition as described herein. In some embodiments, the disease is cancer. In some embodiments, the disease is breast cancer. In some embodiments, the subject is a human or non-human mammal. In other embodiments, non-human mammals include, but are not limited to, livestock and pets, such as ovine, bovine, porcine, canine, feline and murine mammals, as well as simian and non-human primate mammals. In one embodiment, the subject is human. In another embodiment, the subject is a patient.

In other aspects, the present invention provides a method for preventing or disrupting association of IL-1RAcP with an ST2/IL3 complex at a surface of a cell, the method comprising contacting ST2/IL3 complex with a composition as described herein. In some embodiments, the cell is an MDSC. In some embodiments, the method if an in vitro, ex vivo, or in vivo method. For example, in one embodiment, the cell is in a cell culture. In another embodiment, the cell is in a subject.

In some embodiments, the compositions described herein can also be used for the treatment of diseases driven by myeloid immune cell inflammation, such as, for instance, colitis, asthma, and type 1 allergic diseases. Moreover, in some embodiments, the compositions described herein can also be used in inhibiting other types of cell signaling in autoimmune diseases.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: Design of MSTOP Scaffolding and Inhibitory Peptides

A multivalent DNA nanoparticle/peptide hybrid molecular modality platform was developed, which functions as a “decoy ligand” that is selective for a protein-protein interaction (PPI) of interest. This platform is smaller than an antibody but is large enough to target all involved PPI hotspots. Importantly, this platform is assembled with synthetic oligonucleotides, which allows control of the molecular construction. To this end, this modality has tremendous potential for versatility because both the scaffold and its bound inhibitory peptides can be modified to exacting specifications. These synthetic decoy receptors were termed as Multivalent Specific Topology-targeting DNA-Origami Peptide Inhibitors (MSTOPs) (also referred to herein as heteromultivalent nucleic acid scaffolds).

A MSTOP prototype is described that targets the IL-33/ST2 signaling axis by mimicking the hotspots with which endogenous IL-1RAcP interacts with this specific IL-1 cytokine complex (FIG. 5). The MSTOP was designed and constructed to exact specifications to mimic how IL-1RAcP docks with IL-33/ST2. The MSTOP is biologically active in vitro and in reporter gene cell models. The prototype specifically inhibits IL-33 signaling transduction but does not significantly interfere with IL-1β signaling transduction, further suggesting that the prototype MSTOP is selective for IL-33 signaling by mimicking IL1RAcP residues required to orient to IL-33/ST2.

### IL-33 Ternary Complex Modeling

The IL-33 ternary complex was modeled for illustrative purposes, based on the human IL-1β ternary complex with IL-1RAcP, using the Matchmaker tool in Chimera X software. The Chimera search model was PDB #5V14 (murine IL-33 ternary complex), and PDB #4KC3 (human IL-33/ST2 binary complex). PBD #4DEP (Human IL-1β ternary complex) was used for the IL-1RAcP.

### Oligonucleotide Design

3WJ structure, scaffold sequence, and corresponding complimentary staple strands were designed using Tiamat software. Staple specificity for their respective complimentary regions to the scaffold was confirmed with Nucleotide BLAST (ncbi.gov). All oligonucleotides were manufactured by Integrated DNA Technologies (IDT), Inc. (Coralville, IA). Lyophilized oligos were reconstituted in ultrapure, DNase free water to a stock concentration of 500 μM and stored at −20° C. until use. Staple strands were later modified with DBCO moieties on their 3′ ends to build the final MSTOP molecule (IDT, Inc).

### DNA Nanoparticles Folding and Validation

DNA Nanoparticles folding reaction was prepared by combining all oligo components to a concentration of 10 μM with folding buffer (60 mM Tris, 20 mM Acetic Acid, 2 mM EDTA, 12 mM MgCl₂), PBS, and ultrapure water. Reactions were heated to 95° C. and slowly cooled over multiple cycles to 4° C. for 2 h. Accuracy and efficiency of folding reaction and structural stability in PBS with 1% DMSO experimental buffer was carried out at 4° C. at various increments up to 72 h and determined by 4% agarose gel electrophoresis. Thermostability was determined by placing folded 3WJ in a thermocycler and incrementally increasing temperature 0.5° C. every 3 m from 4° C. up to 95° C. to determine Tₘ.

### Dynamic Light Scattering

MSTOP molecules were characterized via DLS, using a Malvern Instruments Zetasizer Nanoseries (Malvern, United Kingdom). Samples were diluted in 0.1× PBS to minimize aggregates to 2 μM then serially diluted 1:2 to 62.5 nM. Samples were assessed at 25 C and 37 C.

### Proteins and Inhibitory Peptides

Recombinant ST2 with truncated FLAG (DDDDK) tag (Cat. #10105-HCCH), His-tagged IL1RAcP (Cat. #10121-H08H), IL-33 (Cat. #10368-HNAE), IL-1β (Cat. #10139-HNAE), and FLAG-tagged SIRT5 (Cat. #19202-H22E) were purchased from Sino Biologicals (Wayne, PA). ST2 and IL1RAcP were expressed in HEK293 cells and included the extracellular domains, K19-F328 and S21-E359, respectively. IL-33, IL-1β, and SIRT5 were expressed in E. coli and included the residue sequence of the cleaved mature proteins, S112-T270, A117-S269, and R2-S310, respectively. Lyophilized ST2, and IL1RAcP were reconstituted in ultrapure (18.5 MΩ) water to a concentration of 0.5 mg/mL (5.6 μM) and stored at −80° C. until use. SIRT5 was reconstituted to 0.2 mg/mL (2.87 μM). To prevent oxidation, IL-33 was reconstituted in 10 mM β-mercaptoethanol (Sigma-Aldrich) to a concentration of 0.1 mg/mL and IL-1β was reconstituted to 0.250 mg/mL. All peptides were purchased from Peptide 2.0 (Chantilly, VA) and designed based on IL1RAcP hotspot residues known to interact with IL-33/ST2 and surrounding residues based on the canonical sequence of IL1RAcP (Uniprot #Q9NPH3). All peptides were modified with acetylated N-terms and amidated C-terms to optimize stability and cyclic peptides cyclized via thioether or disulfide bond. Candidate peptides for the final molecule were also modified with an azido-Lysine and additional N-term alanine or cysteine, to provide the azide group for copper free click chemistry and maintain cyclization. Peptides were stored at −20° C. in lyophilized aliquots of 100 μg or 200 μg and −80° C. after reconstitution in sterile DMSO to a concentration of 10 mM.

### Pulldown Assay

Peptide screening for inhibitory activity was determined with immunoprecipitation pulldown assay using Magnetic Agarose beads coupled with rat α-FLAG antibodies, clone L5 (Pierce, Cat. #A35797). 1.0 μg each of ST2 and IL1RAcP, and 2.0 μg of IL-33 were combined in a reaction buffer of PBS and 5 mM β-mercaptoethanol (BME) (Sigma-Aldrich) to achieve approximate 1:1:1 stoichiometry, based on predicted molecular weights. Inhibitory peptides reconstituted in DMSO were added at a concentration range of 10 μM to 100 μM. 100 μL reaction mixtures were incubated for 25 m in low retention 1.5 mL tubes at RT with gentle rotation. Supernatants were then discarded, and beads were gently washed once with 100 μL ultrapure water. Beads were then eluted in 30 μL of 2× Laemmli buffer with BME (BioRad, Cat. #1610737) at 100° C. for 5 m. Collected samples were diluted to 1× with 30 μL of cold PBS, loaded onto a 4-20% Tris-Glycine gel, and electrophoresed for 1.5 h at 120 volts in SDS-PAGE running buffer. ST2 and IL-33 capture was determined with silver staining (Pierce, cat. #24612) as per the manufacturer's protocol. IL1RAcP trimerization was determined via western blotting. Samples were transferred onto a nitrocellulose membrane for 2 h at 250 mA. Membrane was blocked for 1 h at 4° C. in 20 mL of PBS with 0.1% Tween 20 (PBST) and 5% milk. Blocking solution was then spiked with 20 μL (1:1,000) of mouse anti-human IL1RAcP (ProMab, clone 6D3H5) and incubated ON at 4° C. Membrane was washed 3× with PBST then incubated for 2 hr at 4° C. in 10 mL of PBST with 1 (1: 10,000) of Goat anti-mouse 2° antibody (Invitrogen, cat. #62-6520). Membrane was then washed 2× with PBST, and then a final wash with PBS. Membrane was imaged with horseradish peroxidase substrate (ThermoFisher, Cat #34075) and imaged under UV chemiluminescence (Azure Biosystems; Dublin, CA).

### Cell Culture

HEK-Blue® IL-33 (Invivogen, Cat. #hkb-hil33) and IL-1β cells (Invivogen, Cat #hkb-il1bv2) were cultured in T25 and T75 flasks (NUNC, Cat. #156367 and #156499) in DMEM media supplemented with L-glutamine (Gibco, Cat. #11995-065), 10% Fetal Bovine Serum (Gibco, Cat. #10437-028), 1% Penicillin/Streptomycin (Gibco, Cat. #15140-148), and 0.5% Normacin® (Invivogen, Cat. #ant-hr-1). IL-33 cell growth media also included 1 mL of 250× Selectin Cocktail® (Invivogen, Cat. #hb-sel) and IL-1β cell growth media included 100 μg/mL of Zeocin® (Cat #ant-zn-05). Cells were maintained at 37° C. with humidity and 5% CO₂ and were passaged upon reaching 80-85% confluency. To ensure assay consistency, passage number was kept low (<20) to maintain stable ST2 and IL-1RI expression.

### MSTOP Construction

Peptides were hybridized to the DNA scaffold via copper-free click chemistry. A 1:10 ratio of oligonucleotides modified with DBCO to peptide with azidolysine were incubated at a final concentration of 100 μM and 1 mM, respectively, in ultrapure water either for 2 h at RT or overnight at 4° C. Following incubation, reaction mixtures were combined and added with previously described folding buffer, PBS, and water to a final concentration of 10 μM for the DNA components. Reactions were then placed in a thermocycler with a modified folding reaction with a maximum temperature of 75° C. to prevent peptide degradation and slowly cooled to 4° C. over the course of 2 hours. PCR product was then desalted and concentrated with 3 k MWCO centrifugal filter (Millipore) to remove unreacted peptides and oligonucleotides, quantified via NanoDrop, and resolved on 16% TBE-PAGE to ensure complete folding. Final preparations were incubated with 40 μM DAPI at room temperature (RT) for 30 m to prevent DNA scaffold degradation and further desalted. Protease Inhibitor cocktail (ThermoFisher, Cat. #1860932) was also added to final preparation to protect peptides.

### Crosslinking Assay

MSTOP dose curves were performed with a crosslinking assay. Recombinant IL-33, ST2, and IL-1RAcP were combined as described above and incubated at RT for 30 m in PBS or treated with 10 μM of 3WJ scaffold, WT peptides, or MSTOP in a dose range of 10 μM to 1 nM (1:10 serial dilution). Following incubation, 1 μL of 2.5 mM Disuccinimidyl suberate (DSS, 83 μM) homobifunctional crosslinker was added and reactions were incubated for 1 additional at RT., then quenched with 1 μL of 1 M Tris-HCl. Samples were then prepared for electrophoresis.

### SEAP Assay

IL-33 and IL-1β signaling activation was assessed based on the expression of a SEAP reporter gene. Flasks were washed 2× with prewarmed, sterile PBS (Gibco), scraped in fresh media, and spun down for 5 m at 250×g. Pellet was resuspended in 5 mL test medium (DMEM, 10% heat inactivated FBS, and 1% P/S), cells counted, and concentration adjusted to 2.8×105 cell/mL. 180 μL (˜50,000 cells/well) were seeded in a 96 well, tissue culture treated, flat bottom plate (Costar, Cat. #3595). For IL-33 cells, treatment with 100 pg/mL of IL-33 alone served as a positive control and 100 pg/mL IL-1β served as a negative control. For IL-1β cells, 250 pg/mL of IL-1β alone served as positive control and 250 pg/mL IL-33 served as negative control. 20 of sample containing IL-33 or IL-1β and either peptides or MSTOP molecule were added to wells in a 1:10 serial dilution from 10 μM to 1 pM and the plate incubated for 6 h. After incubation, SEAP reporter gene phosphatase activity was measured by adding 20 μL of supernatant from each well to 180 μL of QuantiBLUE substrate (Invivogen, Cat #rep-qbs) and incubated an additional 30 min at 37° C. Colorimetric absorbance was measured at 620 nm. Data was normalized first to negative controls to account for possible nonspecific stimulation and phosphatase activity from serum and positive controls were set to 100% SEAP activity.

### Surface Plasmon Resonance (SPR)

SPR experiments were carried out on an OpenSPR instrument (Nicoya). Gold nanoparticle sensor chips (Nicoya) were functionalized by incubation in a solution of ethanol and 10 mM 11-Mercaptoundecanoic acid (MUDA) at RT for a minimum of 48 h, then stored in ethanol at 4° C. until use. Chips were activated with a solution of 200 mM EDC and 50 mM NHS for 3 m at RT. 100 μL of Biotinylated BSA (0.5 μg/mL) was then covalently immobilized onto the chip for at least 5 min via standard EDC/NHS chemistry. Chips were washed with ultrapure water, dried with air, and loaded onto the instrument. Instrument was allowed to run for 5 min at 200 μL/min to establish the base line, reduced to 150 μL/min, and loaded with 200 μL Glycine-HCl (pH 1.8) to remove any non-covalently bound Biotin. 200 μL of streptavidin (1.0 mg/mL) was then loaded and injected in the flow cell at 20 μL/min, followed by another Glycine-HCl injection to remove any excess. 50 μg/mL of biotinylated rat α-FLAG mAb (Invitrogen, cat #MA1-91878-BTIN; clone L5) was added twice at a rate of 20 μL/min. For the ST2/IL-33 KD curve, FLAG-tagged ST2 was sent over channel 2 at 20 μL/min followed by FLAG-tagged SIRT5, which was served as a control, over both channels at the same rate. A range of IL-33 from 50 nM to 500 fM, was then sent over both channels at 30 μL/min and allowed to dissociate for 3 m. The surface was regenerated between each IL-33 concentration with 10 mM Glycine-HCl in running buffer (1× PBS w/0.005% Tween 20, pH 7.4). For the IL1RAcP curve, sensor chip preparation was as previously described. ST2 was sent over both channels, followed by 50 nM IL-33 on channel 2 only. A range of IL1RAcP concentration from 10 nM to 1 μM were sent over both flow cells and allowed to dissociate for 3 m. The same protocol was used to assess the K_{D} constants for the individual inhibitory peptides, as well as the final molecule. Data was analyzed with TraceDrawer software to a 1:1 fit model.

### FRET stability Assay

The stability assay with FRET was performed as in Oktay et al.⁴³ Briefly, the MSTOP scaffold were assembled using a Fluorescein (3′-FAM donor)-modified oligonucleotide or with one FAM donor-modified oligonucleotide and one TAMRA (5′-TAM acceptor) along with the non-modified oligonucleotide (Table 1). Fluorescently labelled MSTOP particles were then treated with PBS or 20% human serum, and the FRET signal was recorded for 12 hours using a Tecan Safire Microplate Reader at an excitation wavelength of 455 nm and emission reading from 505 nm to 700 nm. At the end of the assay, DNase was used to induce complete degradation of the nanoparticles and serve as reference. The FRET efficiency (E) was determined using the following equation:

$$FRET efficiency ( E ) : \frac{{I}_{D} - {I}_{DA}}{{I}_{D}}$$

where I_{D} is the intensity of the donor dye (520 nm wavelength) in the MSTOP with FAM alone and I_{DA} the intensity of the donor dye in MSTOP construction containing the FAM/TAM pair.

The percentage of nanoparticles remaining intact (θ) was then determined using the following equation:

$$θ = \frac{( E - E min )}{( E max - E min )}$$

where E is the FRET efficiency, Emin is the FRET efficiency calculated after DNase treatment, and Emax is the initial FRET efficiency at time 0.

### Results/Discussion

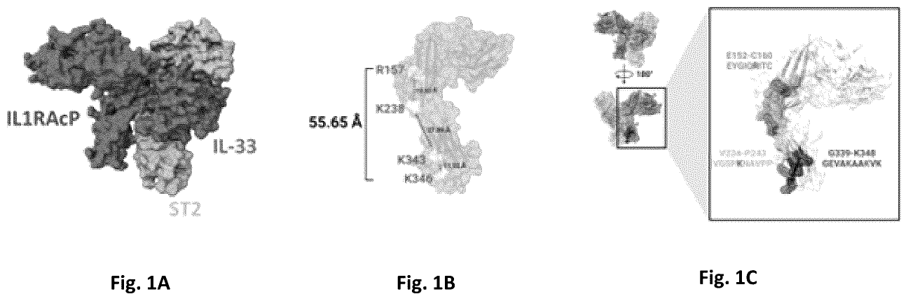

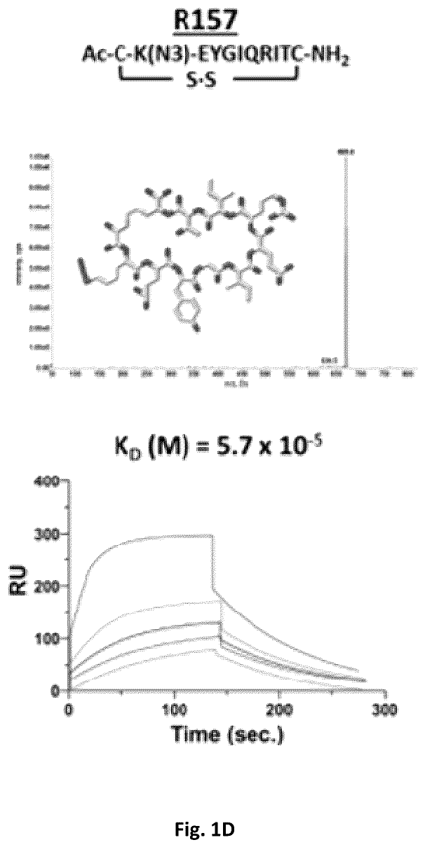

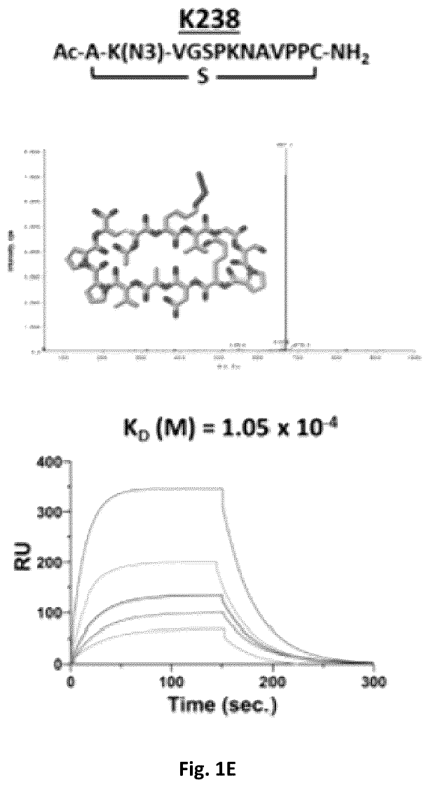

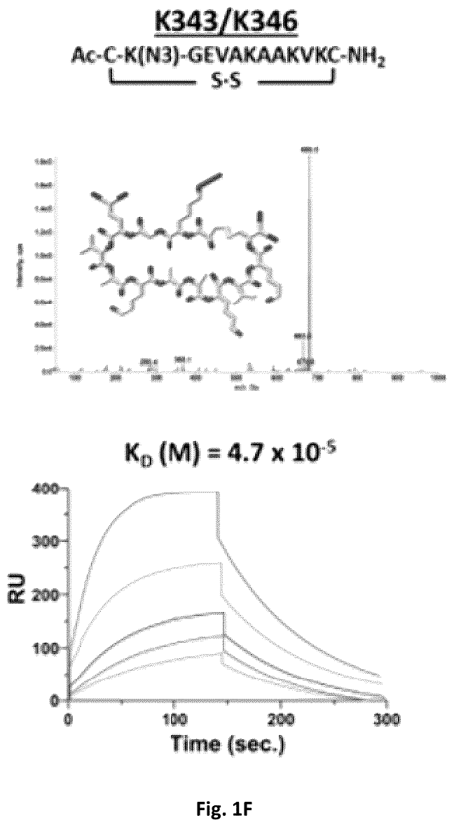

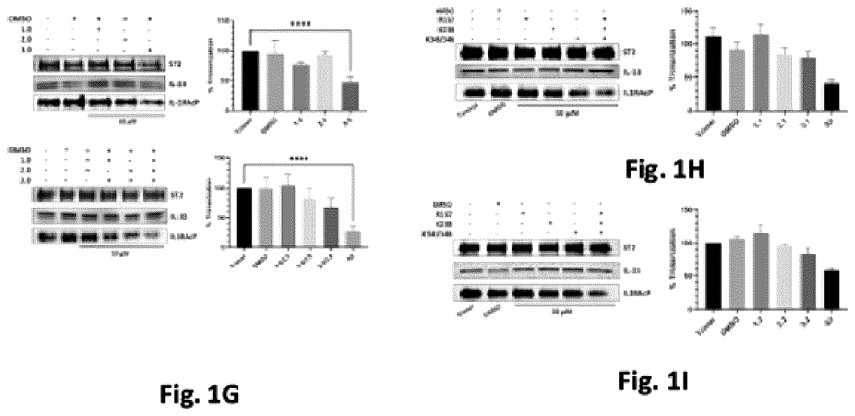

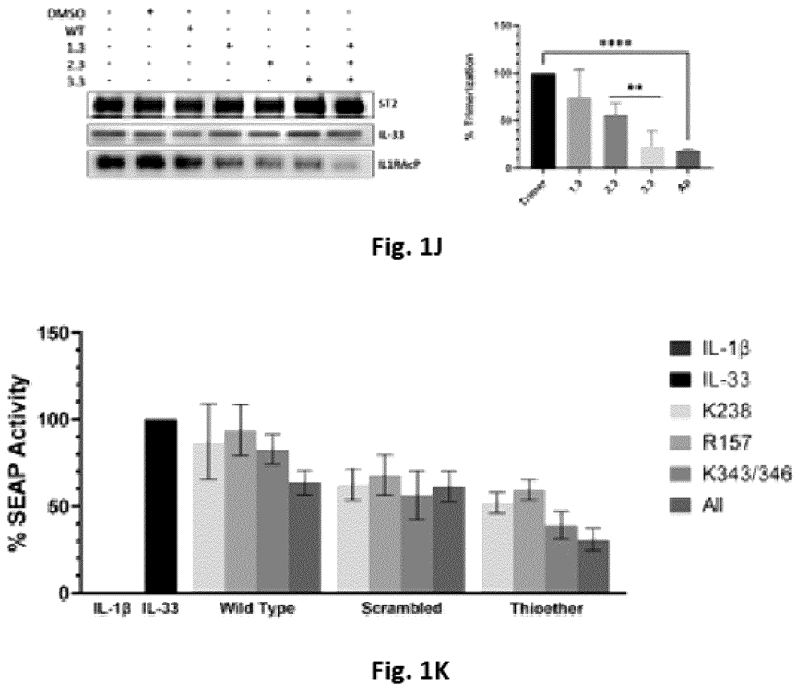

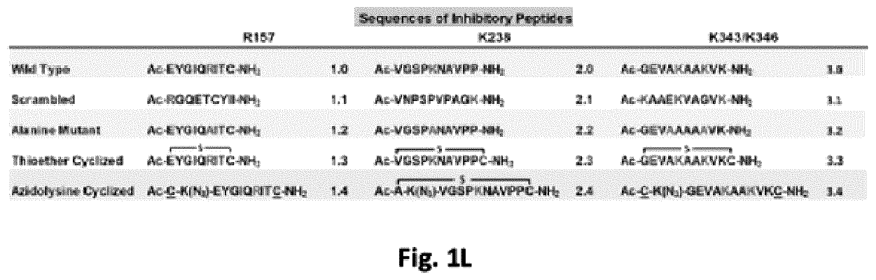

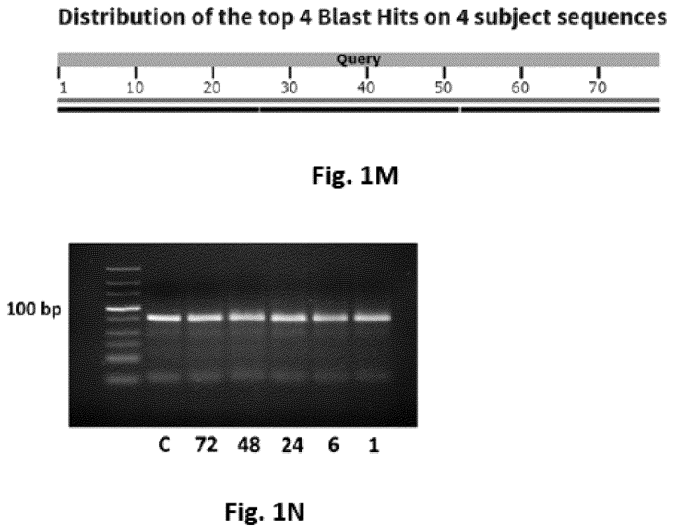

A high-affinity binary complex of ST2/IL-33 (K_{d}=26 pM) must interact with co-receptor IL-1RAcP to form a transient ternary active signaling complex (FIG. 1A). Disruption of this ternary complex, without disrupting the primary cytokine/receptor binary complex, however, is sufficient to abrogate the downstream cytokine mediated signaling. Initial design of the MSTOP prototype first required elucidation of the surface area where IL-1RAcP interfaces with the IL-33/ST2 heterodimer, including the length of PPI interface, hotspot residues where direct contact is made, and distance between each hotspot residue (FIG. 1B). The four hotspots associated with IL-33 signaling stabilization (R157, K238, K343, and K436) were shown to be unique to IL-1RAcP docking to IL-33/ST2, and that IL-1RAcP recruitment to IL-1 β/IL1RI employed different hotspots. See Günther, S. et al., “IL-1 Family Cytokines Use Distinct Molecular Mechanisms to Signal through Their Shared Co-Receptor,” Immunity, 47(3), 510-523.e4 (2017), which is incorporated herein in its entirety. Based on these insights, three small (≤10 aa) inhibitory peptides (FIG. 1L) were then designed based on these hotspots and the surrounding residues in the canonical sequence for the human IL-1RAcP extracellular domain (FIG. 1C). Peptide dissociation constants (K_{D}) were evaluated with SPR. As expected, individual peptides involving partial hotspots are monovalent and have low affinity between 47-105 μM (FIG. 1D-F). The bioactivity of linear, wild-type peptides (WT) was validated and compared to equimolar concentrations of simply modified peptides, such as truncations and peptides that had been cyclized either via a thioether or disulfide bond (FIGS. 1G-1J). The most significant inhibition of ternary complex formation was observed with cyclized variants and when all three peptides were present in solution in both, in vitro and in an IL-33 reporter cell line model (FIG. 1K).

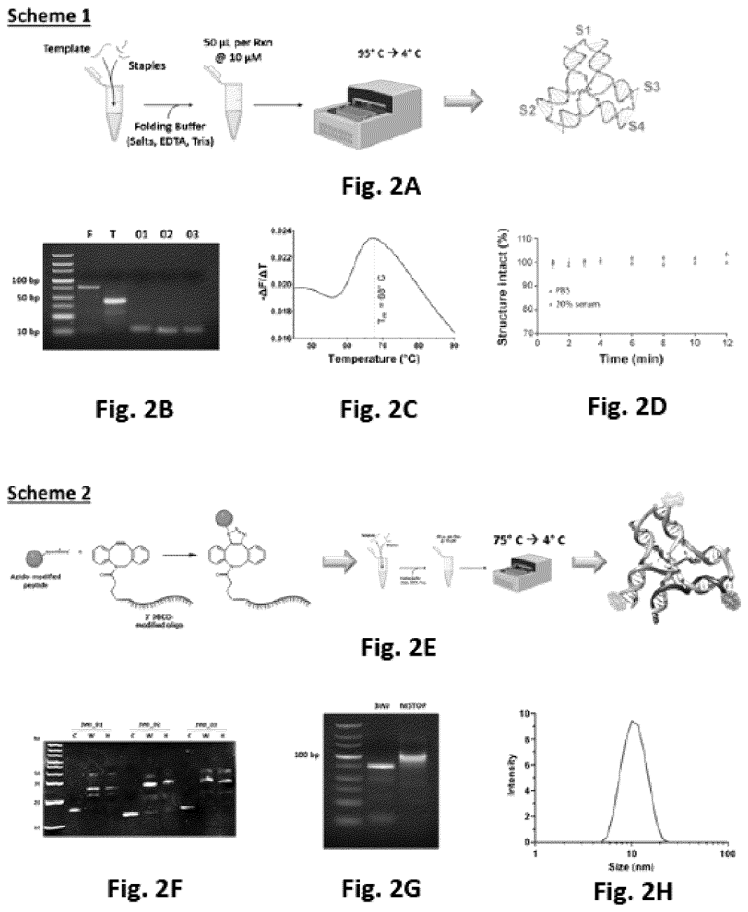

DNA nanotechnology and specifically the tile assembly method is leveraging the unique properties of DNA molecules such as sequence complementarity to enable self-assembly of multiple synthetic oligonucleotides into a wide array of discrete 2D and 3D nanoparticles. See, e.g., Lin, C. et al., DNA Tile Based Self-Assembly: Building Complex Nanoarchitectures, Chemphyschem Eur. J. Chem. Phys. Phys. Chem., 7(8), 1641-1647 (2006); Chandrasekaran, A. R. and Zhuo, R. A, ‘Tile’ Tale: Hierarchical Self-Assembly of DNA Lattices, Appl. Mater. Today, 2, 7-16 (2016). The structural fidelity of assembly at the nanoscale level associated with and the ease of bioconjugation of DNA, allow synthesis of hybrid nanoparticles with prescribed structural features that can mimic the organization of interaction domains in PPIs. Based on the measurements of the IL-1RAcP docking interface, a basic 3-way junction (3WJ) DX-tile was designed to span its length of 56 Å, with a final distance of 59 Å between scaffold “arms” (FIG. 2A). The 3-way junction is assembled with 4 strands (Table 1) at 10 μM in PBS using a slow annealing protocol. Folding was achieved and verified with electrophoresis. It was observed that the scaffold folded as expected with minimal unreacted oligo components (FIG. 2B). Melting temperature of 68° C. was determined by −ΔF/ΔT fluorescence (FIG. 2C). Furthermore, FRET experiments conducted in 20% human serum demonstrated that the scaffold remains stable for at least 12 hours, which appears sufficient for the rest of our study and would be ideal for future in vivo studies (FIG. 2D).

### Example 2: Construction and Characterization of MSTOP Prototype Molecule

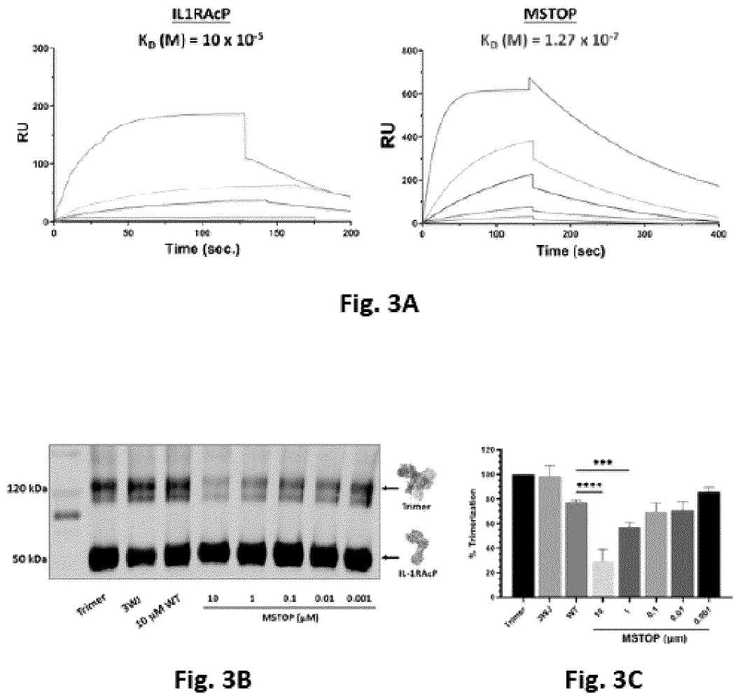

For the final construction of the MSTOP prototype, cyclized inhibitory peptides were designed with an azide-modified lysine (FIG. 2E, FIG. 1L) and oligo staple strands were modified with DBCO on their 3′ ends (FIG. 3B, Table 2), to allow for hybridization via copper-free click chemistry. Each of the 3 oligo staples were incubated in a 1:10 oligo to peptide ratio for 2 hours at RT or overnight at 4° C. This protocol produced complete labelling of the oligos (FIG. 2F). Hybridized staples were then combined with template, previously described salts and buffers, then folded. To prevent possible degradation of the peptides, the folding protocol was modified to have a maximum temperature of 75° C., instead of 95° C. (FIG. 2E). Correct and complete folding of the final prototype structure was confirmed as compared against scaffolding alone with the modified protocol (FIG. 2G), which indicated that the final MSTOP had both increased in molecular weight as expected compared to the scaffold alone and showed no significant side products. Size exclusion was used to purify the MSTOP from excess peptides and oligos. The purified MSTOP was examined via DLS to confirm the expected size distribution. As shown in FIG. 2H, the MSTOP has a hydrodynamic radius of just over 100 Å, as expected, with a suitable tight size distribution. Protease inhibitor cocktail was also added to the final sample to prevent peptide degradation until use in bioactivity assays. Following confirmation that the designed MSTOP has folded as expected and was of suitable purity, its activity was evaluated both in-vitro and in a cell-based model system.

### Example 3: Validation of MSTOP Bioactivity and Specificity

To determine if the DNA scaffolding resulted in a molecule with affinity suitable to outcompete the native IL-1RAcP, SPR was utilized to compare the K_{D} of both to the binary ST2/IL-33 complex. The native receptor has a K_{D} value of 10 μM illustrative of the fact that the interaction is weak and transient (FIG. 3A). In contrast, the MSTOP demonstrated an increased affinity of 195 nM, an almost 100-fold increase compared to the individual peptides and IL-1RAcP (FIG. 3B). The prototype MSTOP was finally assessed for bioactivity via prevention of IL-33/ST2 signaling activation. The MSTOP was first tested in vitro with recombinant ST2, IL-33, and IL-1RAcP incubated together in its endogenous 1:1:1 stoichiometry for 30 minutes. Reactions were then treated with DSS homobifunctional NETS-based crosslinker (final concentration ˜85 μM) to tether formed trimers. Trimer resolved at the predicted 120 kDa in semi-native conditions, while unbound IL-1RAcP resolved at 50 kDa. Quantification of IL-1RAcP at 120 kDa was normalized against total lane IL-1RAcP input, with the untreated trimer control representing 100% trimerization. Treatment with 10 μM of the 3WJ backbone without peptides and with 10 μM of the three cyclized peptides in solution were used as controls to assess MSTOP performance. Treatment with 10 μM of MSTOP resulted in an average of 70% reduction in trimer formation, significantly more inhibition than the equimolar concentration of free peptides in solution (˜20%). Importantly, it was observed that treatment with 1 μM of MSTOP resulted in approximately 40% reduction of trimer formation and the 100 nM MSTOP treatment resulted in similar inhibition to the 10 μM of WT peptide treatment, a 100-fold increase in efficiency that suggests the increased avidity of the MSTOP allowed it to outcompete IL-1RAcP (FIG. 3B-C). Repetition of this experiment showed statistically significant reduction of trimer-associated IL-1RAcP band density as compared to equimolar treatment of the cyclized peptides without the scaffold. Additionally, the scaffold without peptides showed no inhibitory activity.

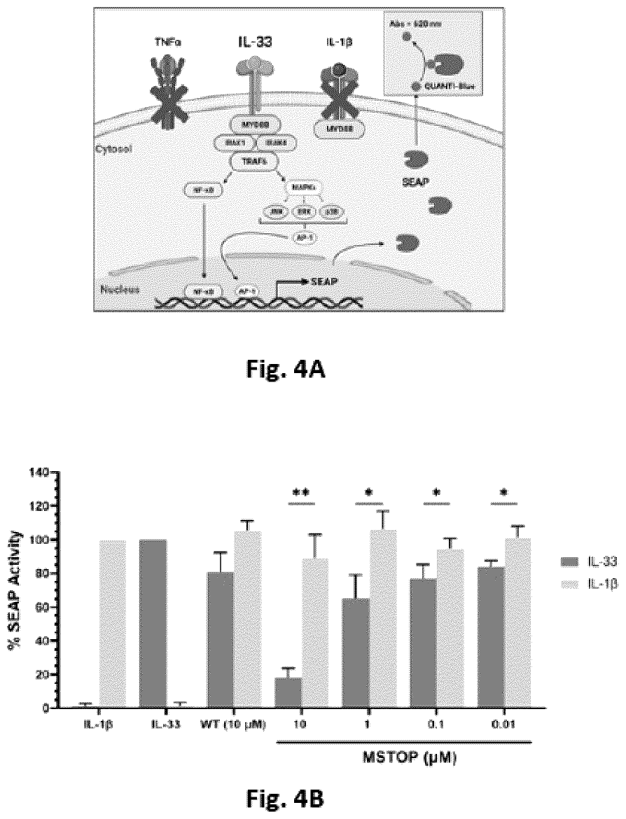

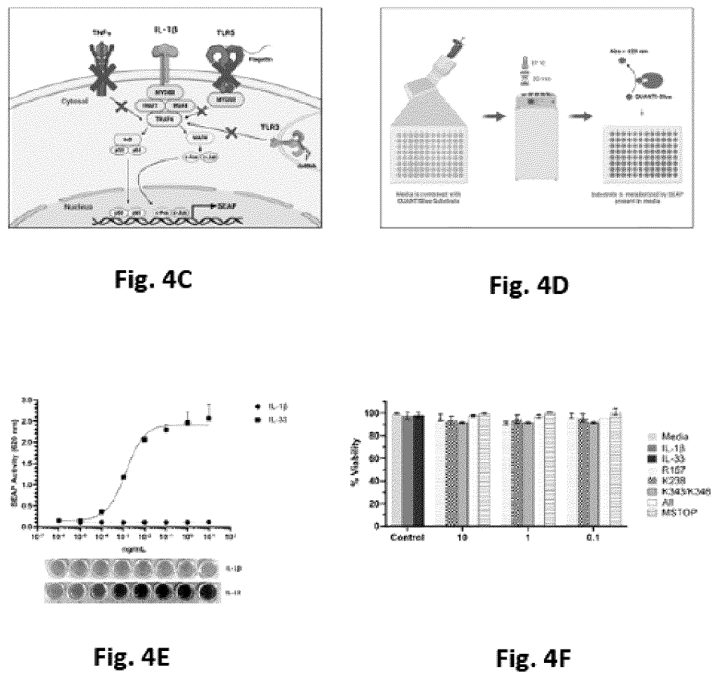

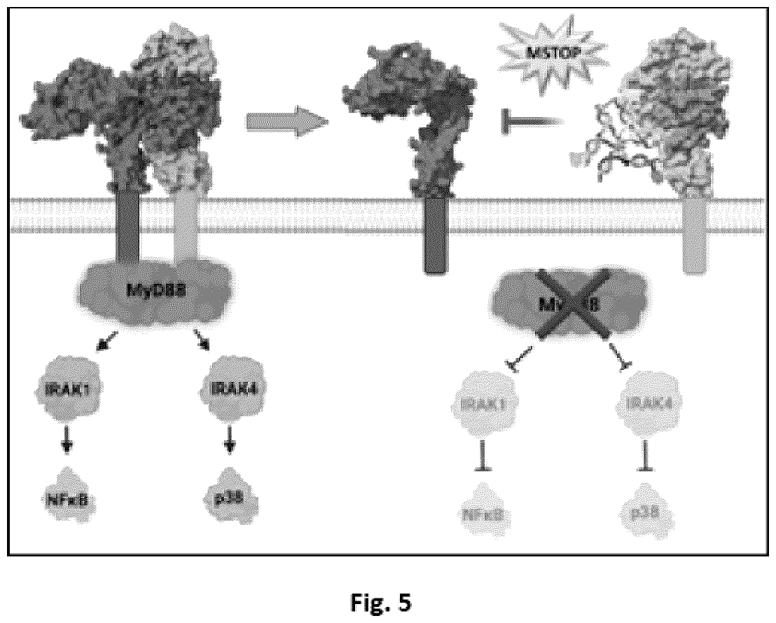

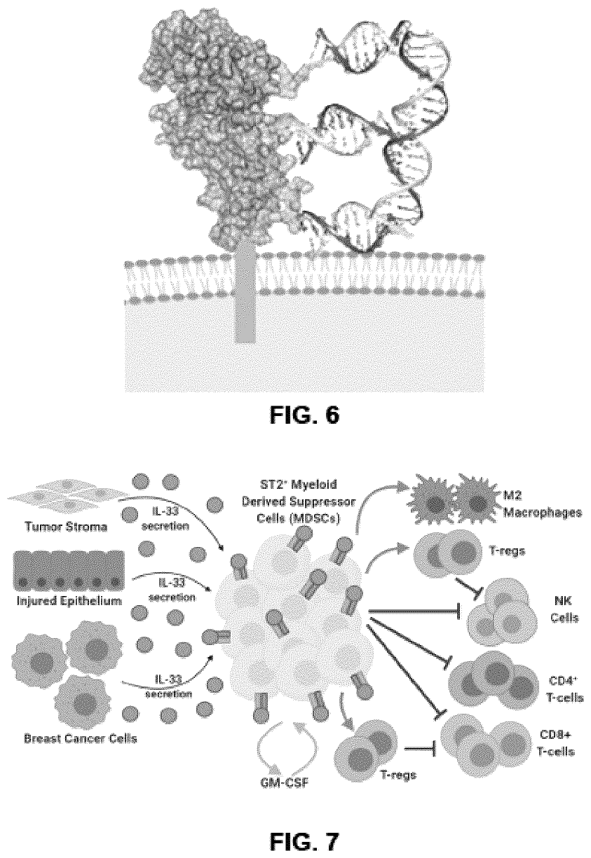

MSTOP bioactivity was determined in a reporter gene cell model. HEK-Blue cells are derived from the HEK293 immortal cell line, and have a SEAP reporter gene cloned at the promoter region of ORFs associated with NFκB and AP-1 pathway gene products. Reporter cells for IL-33 signaling activation are stably transfected to express ST2, while the IL-1β reporter cell counterparts endogenously express IL-1RI. To prevent nonspecific activation of the NFκB and AP-1 pathways and SEAP expression, IL-1RI and TFNRI receptors were suppressed in the IL-33 cells (FIG. 4A) and genes for TLR3, TLR5, and TFNRI, which can also induce the NFκB/AP-1 pathways, are knocked out in the IL-1β cells (FIGS. 4C-4F). Reduction of IL-33 signaling transduction was assessed in IL-33 reporter cells stimulated with 100 pg/mL of IL-33 and either treated with media, 10 μM of the three WT peptides, or a concentration of MSTOP in a dose range from 10 μM to 10 nM. IL-33 reporter cells stimulated with IL-1β served as a control for nonspecific SEAP expression. Since nucleic acids are vulnerable to degradation by endogenous nucleases present in serum (FIG. 2D) the MSTOP was treated with DAPI to protect the 3WJ backbone, a previously reported stabilization strategy. See, e.g., Chandrasekaran, A. R., et al., “Nuclease Resistance of DNA Nanostructures,” Nat. Rev. Chem., 5(4), 225-239 (2021); Hahn, J. et al., “Addressing the Instability of DNA Nanostructures in Tissue Culture,” ACS Nano, 8(9), 8765-8775 (2014). After six hours, IL-33 signaling was quantified by absorbance at 620 nm, based on the metabolism of QUANTI-Blue substrate by expressed SEAP secreted into the media. Remarkably, there was a similar reduction in IL-33 signaling in the cells treated with 10 μM MSTOP to the in vitro experiments, and significant reduction in IL-33 induced SEAP expression (˜40%) was even observed in cells with the 1 μM treatment (FIG. 4B). Furthermore, treatment with 10 μM MSTOP did not decrease cell viability as measured by CellTiterGlo assay (FIGS. 4C-4F).

To establish MSTOP selectivity for the IL-33/ST2 axis, IL-1β reporter cells were stimulated with 250 pg/mL of IL-1β and treated with either media, 10 μM of WT peptides, or the same concentrations of MSTOP as the IL-33 cells. Negligible reduction in SEAP expression was observed in all conditions, including the 10 μM MSTOP treatment, suggesting that MSTOPs can be designed to select for a particular PPI with minimal perturbation of structurally similar PPIs within the same protein family (FIG. 4B).

### Discussion

Targeting PPIs to modulate aberrant intra- and extracellular functions is an essential goal for future pharmacologics. Nevertheless, PPI drug development thus far has proven quite challenging due to large contact areas, complicated 3D topology, or flat interfaces. Consequently, small molecules may have low potency and specificity, because they interfere with only a subset of the multiple contact points spanning large PPI interfaces; thus, touchpoints outside the binding region of a small molecule may still associate. In contrast, large bulky inhibitory biologics do not present a 3D footprint that is specific enough for the selectivity required to minimize perturbation of proximal, but irrelevant PPIs. To this end, the MSTOP modality was developed as a solution to expand the repertoire of druggable PPI targets. Importantly, the MSTOP molecule is fully synthetic, providing exquisite design control and utilizes existing knowledge of PPI interfaces and high energy residue touchpoints, allowing for recognition of extremely subtle conformational changes that may alter the interface topology to facilitate a PPI. Furthermore, synthesis only requires routine techniques and instruments, and is easily executed in most molecular biology laboratories, meaning this platform has potential for versatility as synthesis can be modified based on the PPI to be targeted, providing a range of therapeutics that are “made to order” for a given target.

The MSTOP protein-DNA hybrid molecule tethers multiple protein ligands recognizing different nearby targets onto a DNA scaffold, creating a heteromultivalent ligand. One of the notable results of this study is a dramatic 100-fold improvement in the functional affinity of less than 200 nM (FIG. 3A) for the MSTOP multivalent ligand, compared to the individual non-tethered protein ligands, which had dissociation constants in the mid to high μM range (FIG. 1D-F). Pharmacologic strategists have previously recognized that tethering different antibody fragments, or even low molecular weight compounds, will generate heterobivalent ligands that exhibit markedly increased affinity (i.e. avidity) and improve target residence time (low off-rate). This is because binding of one ligand forces the second tethered ligand to stay in proximity to its corresponding target site. This ‘forced proximity’ greatly promotes initial binding and rebinding, once dissociated, to that site. In addition, rebinding will also take place with higher probability when the diffusion of freshly dissociated tethered ligands is restricted to remain in proximity to its target. Vaquelin et al. employed differential equation-based simulations to study the impact of heterobifunctional ligand tethering on functional affinity. Tethering incurs steep saturation curves and increases the target residence time. Simulations of heterobivalent ligand binding kinetics, with each individual tethered ligand possessing an affinity of 10⁻⁶ M for its own target, compared to a monovalent interaction, demonstrates greater than 100 fold improvement in the dissociation t_{1/2} values. This is logical because it is highly improbable for multiple tethered ligands to dissociate simultaneously. These simulations of improved affinity engendered by multivalency are in keeping with our experimental MSTOP data, providing a strong theoretical basis for our observations.

Finally, multivalency provides PPI specificity, which determines downstream biochemical events. The IL-1β and IL-33 cytokine receptor complexes both require IL-1RAcP recruitment to stabilize the docking site and induce the subsequent signaling cascade. While IL-1RAcP orientation with these ternary complexes appears nearly identical based on solvation via crystallography, IL-1RAcP employs unique hotspots with which to interact with these complexes. By mimicking the hotspot residues unique to IL-1RAcP docking with IL-33/ST2, the MSTOP molecule selects for this particular heterodimer with minimal interaction with the structurally similar IL-1β/IL-1RI (FIG. 4B).

Since both efficacy and selectivity of MSTOP have been determined in vitro and in reporter cell models, further optimization of the MSTOP molecular paradigm must be investigated and confirmed to improve its stability and functional affinity prior to assessing bioactivity in vivo. For example, nucleic acids are vulnerable to degradation by endogenous nucleases. In this study, the MSTOP was treated with DAPI to avoid this degradation so that bioactivity in the cells could be determined. This is not a viable solution going forward, however, and, nuclease resistant nucleic acid mimetics, such as PNA, phosphorothioate, should be explored. Whether functional affinity can be improved upon will also be investigated. Mutations of neighboring residues and amino acid analogs will be considered to further improve the functional affinity of the original MSTOP. Finally, it is necessary to assess efficacy in a more analogous cell model to determine if MSTOP can prevent macrophage polarization into M2, a deleterious pro-cancer phenomenon in solid malignant tumors.

These studies suggest that the MSTOP prototype developed has the tremendous potential to become a specific and effective platform tool for PPI modulation, that can circumvent the limitations of mAbs and other biologicals. Importantly, MSTOP fully synthetic construction lends great versatility, with simple modification to the specifications of a given PPI.

### Example 4: DNA Scaffold Toxicity Analysis

The potential toxicity of the naked DNA scaffold is analyzed. Following the successful assembly of DNA scaffolds, their cytotoxicity is determined using cell viability assays with THP-1 monocyte cell line derived macrophages as previously described. In brief, THP-1 monocytes are cultured in RPMI 1640+10% inactivated FBS. Addition of 10 ng/mL PMA allows for the development of monocyte derived macrophages (MDMs) in 3-5 days. MDMs are treated with the naked DNA scaffold and are examined for viability using CellTiter-Blue (Promega) and CytoTox96 (Promega). CpG-ODN 2216 (known to stimulate pro-inflammatory monocyte polarization) and/or lipopolysaccharide (LPS) act as positive controls. The toxicity results are then analyzed for any potential toxicity and degree thereof of the naked DNA scaffolds.

### Example 6: MSTOP for Breast Cancer Treatment

MSTOPs, such as those described herein (also referred to herein as heteromultivalent nucleic acid scaffolds), are designed that target activated myeloid-derived suppressor cells (MDSCs) in the breast cancer tumor microenvironment. As compared to healthy controls, breast cancer patients are reported to have a 10-fold higher fraction of MDSCs in freshly-isolated peripheral blood mononuclear cells and the presence of high levels of MDSCs in the tumor microenvironment is associated with poor prognosis for cancer patients. MDSCs in the breast tumor stroma are a major cause of tumor immune escape that blocks successful breast cancer immunotherapy and can also reduce the efficacy of CAR-T therapies. MDSCs inhibit the immune cell recognition and the host killing of breast tumor cells via a variety of mechanisms. These mechanisms include cytokine production to recruit additional classes of immunosuppressive cells, blocking of lymphocyte homing to the tumor, production of reactive oxygen and nitrogen species, depletion of metabolites and modulation of adenosine, and expression of negative immune checkpoint molecules (FIG. 7). Moreover, beyond immunotherapy, experimental studies indicate that myeloid suppressor cells modulate a wide variety of key cancer-associated activities that affect virtually all types of cancer therapy.

The complex of interleukin-33, receptor ST2, and co-receptor IL1-RAcP is a therapeutic target to specifically inhibit MDSCs. IL-33/ST2 induced growth and survival of MDSCs in the immune competent murine and human breast cancer microenvironment has been proven to be functionally required for immune invasion. IL-33 is recognized as an “alarmin,” or a damage-associated molecule “DAMP,” generated in the breast tumor microenvironment that attracts and sustains MDSCs to suppress a variety of innate immune functions. This novel cell surface target is specific to functionally active MDSCs in the breast tumor microenvironment and could be targeted MSTOPs, such as depicted in FIG. 12A-FIG. 12B. Because the IL-1RAcP/ST2/IL-33 complex is essential for MDSC immunosuppressive signaling, inhibition could allow for immunotherapeutic treatment of breast cancer.

Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

In sum, while this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

I/We claim:

1. A heteromultivalent nucleic acid scaffold comprising a scaffold strand and at least two staple strands, wherein each staple strand comprises a peptide attached thereto, and wherein each peptide specifically contacts a different region of a protein, thereby binding the multivalent nucleic acid scaffold to the protein.

2. The heteromultivalent nucleic acid scaffold of claim 1, wherein the scaffold strand and/or the at least two staple strands, independently, each comprises DNA and/or RNA.

3. The heteromultivalent nucleic acid scaffold of claim 2, wherein the scaffold strand and/or the at least two staples strands, independently, each is PEGylated.

4. The heteromultivalent nucleic acid scaffold of clam 1, wherein the scaffold strand and/or the at least two staples strands, independently, each comprises peptide nucleic acids (PNA) or phosphorothioate-polymerized nucleotides.

5. The heteromultivalent nucleic acid scaffold of claim 1, wherein at least one staple strand comprises a small molecule .

6. The heteromultivalent nucleic acid scaffold of claim 5, wherein each peptide is attached to its staple strand, independently, by NHS-based coupling via amine groups, maleimide coupling via cysteine residues, carbodiimide coupling via carboxyl groups, copper-free click chemistry, or by complementary nucleic acid overhangs.

7. The heteromultivalent nucleic acid scaffold of claim 1, wherein each peptide is a cyclized peptide.

8. The heteromultivalent nucleic acid scaffold of claim 1, wherein the at least two staple strands comprise a first staple strand comprising a first peptide, a second staple strand comprising a second peptide, and a third staple strand comprising a third peptide, and wherein the first peptide is Ac-C-K(N₃)-EYGIQRITC-NH₂, the second peptide is Ac-A-K(N₃)-VGSPKNAVPPC-NH₂, and the third peptide is Ac-C-K(N₃)-GEVAKAAKVKC-NH₂.

9. The heteromultivalent nucleic acid scaffold of claim 1, wherein the binding of the multivalent nucleic acid scaffold to the protein prevents or disrupts association of the protein with a binding partner by mimicking at least two different protein-protein interaction hotspots with which the protein interacts with the binding partner.

10. The heteromultivalent nucleic acid scaffold of claim 1, wherein each peptide comprises about 5 to about 15 amino acid residues in length.

11. The heteromultivalent nucleic acid scaffold of claim 1, wherein each peptide is capable of preventing or disrupting association of IL1RAcP with an ST2/IL3 complex at a surface of a cell.

12. The heteromultivalent nucleic acid scaffold of claim 11, wherein each peptide, independently, targets position R157, K238, K343, and/or K346 of IL1RAcP (SEQ ID NO: 31).

13. The heteromultivalent nucleic acid scaffold of claim 1, wherein each, independently, comprises the amino acid sequence of any one of SEQ ID NOs: 1-18.

14. The heteromultivalent nucleic acid scaffold of claim 1, wherein the heteromultivalent nucleic acid scaffold comprises one or more sequences as set forth in any one of SEQ ID NOs: 19-30.

15. A composition comprising the heteromultivalent nucleic acid scaffold of claim 1; and a pharmaceutically acceptable carrier.

16. A method for treating a disease in a subject in need thereof, the method comprising:
administering a therapeutically effective amount of the composition of claim 15 to the subject.

17. The method of claim 16, wherein the disease is cancer.

18. The method of claim 17, wherein the cancer is breast cancer.

19. A method for preventing or disrupting association of IL-1RAcP with an ST2/IL3 complex at a surface of a cell, the method comprising:
contacting IL-1RAcP with the composition of claim 15.

20. A peptide comprising the amino acid sequence set forth in any one of SEQ ID NOs: 1-18.
